(19) United States
(12) Patent Application Publication
(10) Pub. No.: US 2011/0207987 A1
(43) Pub. Date: Aug. 25, 2011

(54) Methods And Devices For Delivering Appropriate Minimally-Invasive Extraocular Radiation

(75) Inventors: Paul DiCarlo, Middleboro, MA (US); Eric Meade, Newton, MA (US); Robert Degon, Bellingham, MA (US); Russell J. Hamilton, Tucson, AZ (US)

(73) Assignee: SALUTARIS MEDICAL DEVICES, INC., Tucson, AZ (US)

(21) Appl. No.: 12/917,044

(22) Filed: Nov. 1, 2010

Related U.S. Application Data
(60) Provisional application No. 61/257,232, filed on Nov. 2, 2009.
(60) Provisional application No. 61/376,115, filed on Aug. 23, 2010.

(51) Int. Cl. A61N 5/00

(52) U.S. Cl. 600/3

(57) ABSTRACT

The present invention also features a brachytherapy system comprising a spiral cut tube having a first end and a second end; a radioactive brachytherapy source (RBS) disposed on the first end of the spiral cut tube; and a handle and a generally hollow cannula disposed on the handle, wherein a channel is disposed in the handle aligned with the hollow cannula, and the spiral cut tube and RBS are adapted to slide within the channel and the hollow cannula.

### CROSS REFERENCE

This application claims priority to U.S. provisional application Ser. No. 61/257,232 filed Nov. 2, 2009 and U.S. provisional application Ser. No. 61/376,115 filed Aug. 23, 2010, the specifications of which are incorporated herein by reference in its entirety.

### FIELD OF THE INVENTION

The present invention is directed to minimally-invasive methods and devices for introducing radiation to the posterior portion of the eye for treating and/or managing eye conditions including but not limited to macula degeneration.

### BACKGROUND OF THE INVENTION

Several diseases and conditions of the posterior segment of the eye threaten vision. Age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, and glaucoma are several examples.

Age related macular degeneration (ARMD) is the leading cause of blindness in the elderly. ARMD attacks the center region of the retina (i.e., macula), responsible for detailed vision and damages it, making reading, driving, recognizing faces and other detailed tasks difficult or impossible. Current estimates reveal that approximately forty percent of the population over age 75, and approximately twenty percent of the population over age 60, suffer from some degree of macular degeneration. “Wet” or exudative ARMD is the type of ARMD that most often causes blindness. In wet ARMD, newly formed choroidal blood vessels (choroidal neovascularization (CNV)) leak fluid and cause progressive damage to the retina. About 200,000 new cases of Wet ARMD occur each year in the United States alone.

Brachytherapy is treatment of a region by placing radioactive isotopes in, on, or near it. Both malignant and benign conditions are successfully treated with brachytherapy. Lesion location dictates treatment technique. For the treatment of tumors or tumor beds in the breast, tongue, abdomen, or muscle capsules, catheters are inserted into the tissue (interstitial application). Radiation may be delivered by inserting strands of radioactive seeds into these catheters for a predetermined amount of time. Permanent implants are also possible. For example, in the treatment of prostate cancer, radioactive seeds are placed directly into the prostate where they remain indefinitely. Restenosis of coronary arteries after stent implantation, a non-malignant condition, has been successfully treated by placing a catheter into the coronary artery, then inserting a radioactive source into the catheter and holding it there for a predetermined time in order to deliver a sufficient dose to the vessel wall. Beta emitters, such as phosphorus 32 (P-32) and strontium 90 (Sr-90), and gamma emitters, such as iridium 192 (Ir-192), have been used. The Collaborative Ocular Melanoma Study (COMS), a multicenter randomized trial sponsored by the National Eye Institute and the National Cancer Institute demonstrated the utility of brachytherapy for the treatment of ocular cancers and/or tumors. The technique employs an invasive surgical procedure to allow placement of a surface applicator (called an episcleral plaque) that is applied extraocullarly by suturing it to the sclera. The gold plaque contains an inner mold into which radioactive iodine 125 (1-125) seeds are inserted. The gold plaque serves to shield the tissues external to the eye while exposing the sclera, choroid, choroidal melanoma, and overlying retina to radiation. The plaque remains fixed for a few days to one week in order to deliver approximately 85 Gy to the tumor apex.

Radiotherapy has long been used to treat arteriovenous malformations (AVM), a benign condition involving pathological vessel formation, in the brain. An AVM is a congenital vascular pathology characterized by tangles of veins and arteries. The dose applicable to the treatment of neovascularization in age-related macular degeneration (WAMD) by the devices described herein may be based on stereotactic radiosurgery (SRS) treatment of arteriovenous malformations (AVM). SRS is used to deliver radiation to the AVM in order to obliterate it, and radiation is highly effective for AVM treatment. The minimum dose needed to obliterate an AVM with high probability is approximately 20 Gy. However, small AVMs (<1cm) are often treated with a higher dose (e.g., 30 Gy) because when treating small AVMs, a significant amount of eloquent brain (e.g., brain regions wherein injury typically causes disabling neurological deficits) is not exposed to the high' dose of radiation. The reported SRS doses correspond to the dose received at the periphery of the AVM, while the dose at the nidus (center) may be up to a factor of 2.5 times greater than the reported SRS dose.

The vascular region involved in WAMD is much smaller than even the smallest AVM, thus the effective doses are expected to be similar to the highest doses used for AVM. Studies of irradiation of WAMD have shown that greater than 20 Gy are required, although one study indicates some response at 16 Gy. Without wishing to limit the present invention to any theory or mechanism, the devices described herein for WAMD are expected to be effective by delivering a nearly uniform dose to the entire region of neovascularization or by delivering a nonuniform dose which may vary by a factor of 2.5 higher in the center as compared to the boundary of the region with minimum doses of 20 Gy and maximum doses of 75 Gy. A report using radiosurgery for macular degeneration describes that a dose of only 10 Gy was not effective (Haas et al, J Neurosurgery 93, 172-76, 2000). In that study, the stated dose is the peripheral dose with the center being about 10% greater. Furthermore, the study results were severely plagued by retinal complications.

Without wishing to limit the present invention to any theory or mechanism, it is believed that the devices of the present invention are advantageous over the prior art. For example, since SRS employs external photon beams which easily penetrate the ocular structures and pass through the entire brain, the patient must be positioned such that the beams may be directed towards the macula, making the geometric uncertainties of delivery a few millimeters. The devices of the present invention have geometric and dosimetric advantages because they may be placed at the macula with submillimeter accuracy, and the beta radioisotope may be used to construct the radiation source with predominately limited range.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### SUMMARY

The present invention features minimally-invasive methods and devices for introducing radiation to the posterior portion of the eye for treating and/or managing eye conditions including but not limited to macula degeneration.

The present invention features a spiral cut tube having a first end and a second end, wherein a radioactive brachytherapy source (RBS) disposed on the first end of the spiral cut tube. The spiral cut tube may further comprise a handle and a generally hollow cannula disposed on an end of the handle, wherein a channel is disposed in the handle aligned with the hollow cannula and wherein the spiral cut tube is adapted to slide within the channel and the hollow cannula. The present invention also features a brachytherapy system comprising a spiral cut tube having a first end and a second end; a radioactive brachytherapy source (RBS) disposed on the first end of the spiral cut tube; and a handle and a generally hollow cannula disposed on the handle, wherein a channel is disposed in the handle aligned with the hollow cannula, and the spiral cut tube and RBS are adapted to slide within the channel and the hollow cannula.

In some embodiments, the RBS (e.g., cylindrical, spheres, disc-shaped, annulus-shaped, irregular in shape, etc.) is secured to the first end of the spiral cut tube via a securing means (e.g., welding). In some embodiments a solid shaft is disposed on the second end of the spiral cut tube.

In some embodiments, a visual marker is disposed on the spiral cut tube. In some embodiments, a visual marker is disposed on the solid shaft. In some embodiments, a visual marker is disposed on the RBS. In some embodiments, the visual maker on the spiral cut tube and/or solid shaft and/or RBS is visible from outside the handle or the cannula. For example, a window or aperture may be disposed in the PIG/handle, which allows the markers to be visualized from outside the handle or cannula.

In some embodiments, the spiral cut tube has a cut angle of about 5.12 degrees, between about 4 to 4.5 degrees, between about 4.5 to 5 degrees, between about 5 to 5.5 degrees, between about 5.5 to 6 degrees, between about 6 to 6.5 degrees, less than about 4 degrees, or more than about 6.5 degrees.

In some embodiments, the spiral cut tube is about 2.3 inches in length as measured from the first end to the second end, between about 1 to 2 inches in length as measured from the first end to the second end, between about 2 to 3 inches in length as measured from the first end to the second end, between about 3 to 4 inches in length as measured from the first end to the second end, less than about 1 inch in length as measured from the first end to the second end, or more than about 4 inches in length as measured from the first end to the second end.

In some embodiments, cuts on the spiral cut tube are about 0.02 inches apart, between about 0.005 to 0.01 inches apart, between about 0.01 to 0.02 inches apart, between about 0.02 to 0.03 inches apart, less than about 0.005 inches apart, or more than about 0.03 inches apart. In some embodiments, cuts on the spiral cut tube are about 0.001 inches in width, between about 0.0001 to 0.001 inches in width, between about 0.001 to 0.01 inches in width, less than about 0.0001 inches in width, or more than about 0.01 inches in width.

The present invention also features a brachytherapy device comprising a handle having a radiation shielding PIG for shielding a RBS, wherein at least a portion of the radiation shielding PIG is generally visually clear, transparent, or translucent.

In some embodiments, the handle is constructed from a plastic, a glass (e.g., durable glass such as Gorilla® Glass), or a combination thereof, for example polyetherimide, poly (methyl methacrylate), acrylic polysulfone, polycarbonate, or polypropylene.

The brachytherapy device may further comprise a distal portion for placement around a portion of a globe of an eye, a proximal portion, and an inflection point, which is where the distal portion and the proximal portions connect with each other; wherein the handle is attached to the proximal portion. The distal portion may have a radius of curvature between about 9 to 15 mm and an arc length between about 25 to 35 mm, and the proximal portion may have a radius of curvature between about an inner cross-sectional radius of the cannula and about 1 meter. The handle may be removably attached to the proximal portion, e.g., via a thumb screw attachment mechanism or other attachment mechanism.

The radiation shielding PIG may be constructed from a material comprising a polyetherimide, polysulfone, polycarbonate, or polypropylene. In some embodiments, the radiation shielding PIG is positioned at or near a first end of the handle, at or near a middle portion of the handle, or at or near a second end of the handle. In some embodiments, the PIG has a flat edge. In some embodiments, a visual landmark is disposed on the PIG, wherein the visual landmark functions as a reference point for orientation before or during a surgical procedure.

The radiation shielding PIG may have a generally cylindrical shape, a generally oval shape, or a generally octagonal shape. In some embodiments, the radiation shielding PIG has an outer diameter between about 2.0 and 3.0 cm and/or an inner diameter of less than or equal to about 0.1 cm

The brachytherapy device may further comprise a means of moving a RBS (e.g., a plunger) within the handle. In some embodiments, a control cable system manipulating the means of moving the RBS (e.g., plunger). In some embodiments, the first end of the control cable system is connected to the means of moving the RBS. In some embodiments, an actuator handle is disposed on a second end of the control cable system. In some embodiments, the control cable system comprises a central wire rope (e.g., constructed from a material comprising stainless steel, e.g., stainless steel coated with nylon) surrounded by an outer tube (e.g., constructed from a material comprising polyvinyl chloride). The inner diameter of the outer tube may be lined with fluorinated ethylene propylene, polytetrafluoroethylene, acrylic, or a combination thereof. The brachytherapy device may further comprise a stainless steel tube disposed on a portion of the control cable system near the actuator handle.

The brachytherapy device may further comprise a secondary radiation shield attachable to the handle.

The brachytherapy device may further comprise a marker disposed on the advancing means (e.g., plunger), wherein the marker functions as a reference point for positioning of the advancing means (e.g., plunger). The marker may be able to be visualized from outside the brachytherapy device (e.g., via a window disposed in the handle/PIG). In some embodiments, the marker on the advancing means (e.g., plunger) can be visualized from outside the brachytherapy device when the plunger is in a treatment position. For example, a window may be disposed in the PIG, which allows visualization of the advancing means (e.g., plunger) and/or spiral cut tube and/or RBS. In some embodiments, an aperture is disposed in the handle, wherein the aperture is positioned such that when the means of moving a RBS is positioned in a treatment position the marker is visible through the aperture.

The present invention also features methods of calibrating radioactive brachytherapy source (RBS) placement. The methods may comprise (a) obtaining a radiation shield having an inner cavity; (b) placing a film (e.g., dosimetry film, e.g., GafChromic®) in the inner cavity of the radiation shield, the film having a visual marker disposed thereon; (c) placing a tip of a cannula atop the film, the cannula having a light source disposed at the tip, the light source being aligned atop the visual marker disposed on the film; (d) activating an advancing means disposed in the cannula for a first length of time (e.g., about 2 to 5 seconds, about 5 seconds, about 5 to 10 seconds, more than about 10 seconds, etc.), said advancing means functioning to advance a RBS to the tip of the cannula or to near the tip of the cannula, wherein a reaction on the film occurs due to exposure to the RBS; and (f) analyzing said film. If the reaction on the film occurs on the visual marker of the film the RBS placement is calibrated. If the reaction on the film does not occur on the visual marker of the film the RBS placement is not calibrated. If the RBS placement is not calibrated, the advancing means may be adjusted accordingly.

The radiation shield may comprise a base having a groove disposed in a top surface near a side edge. A lid may be pivotally or removably attached to the base, wherein the lid forms an inner cavity. The lid can move between at least an open position and a closed position respectively allowing or preventing access to the inner cavity. A slot is disposed in the lid at a bottom surface, the slot and the groove align when the lid is in the closed position. Generally, the radiation shield is of sufficient thickness to block passing of beta radiation.

The present invention also features a cannula comprising a light system for emitting light from a tip of the cannula. The light system is constructed from a fiber, wherein the fiber runs along an outside portion of the cannula. The fibers may be constructed from a material comprising poly(methyl methacrylate), glass, the like, or a combination thereof.

In some embodiments, the cannula further comprises a distal portion for placement around a portion of a globe of an eye, the distal portion has a radius of curvature between about 9 to 15 mm and an arc length between about 25 to 35 mm; a proximal portion having a radius of curvature between about an inner cross-sectional radius of the cannula and about 1 meter; and an inflection point which is where the distal portion and the proximal portions connect with each other; wherein the handle is attached to the proximal portion of the cannula.

The fibers of the light system may run along outside the distal portion and the proximal portion of the cannula. In some embodiments, the fiber and outside portion of the cannula together are covered with a heat shrink tube (e.g., polyethylene terephlalate or poly ether ether ketone), for example the fiber, proximal portion, and distal portion are together covered with a heat shrink tube. In some embodiments, the fibers are tacked to the outside portion of the cannula via an adhesive (e.g., UV adhesive).

In some embodiments, the light from the light system is directed at an angle from the tip. In some embodiments, the angle is between about 40 to 50 degrees, between about 50 to 60 degrees, between about 60 to 70 degrees, and/or between about 70 to 75 degrees. In some embodiments, a lens or a reflective material is used to angle the light.

The present invention also features a cannula comprising a sensor for detecting a presence of an RBS at a position within the cannula. The sensor is operatively connected to both a power source and an alert system. Upon detection of the presence of the RBS at the position within the cannula the sensor triggers the alert system to notify a user that the RBS is at the position within the cannula.

In some embodiments, the sensor detects the present of the RBS in a treatment zone. In some embodiments, the sensor activates a light source when the RBS is detected in the treatment zone. In some embodiments, the sensor is an electrical system. For example, in some embodiments, the sensor is a transistor (e.g., a solid-state transistor, a metal-oxide-semiconductor field-effect transistor (MOSFET), etc.). In some embodiments, the sensor is a non-electrical system (e.g., phosphorus).

In some embodiments, other components may be used in place of a transistor in accordance with the present invention. The following non-limiting electrical components may be used: a semiconductor device that may be used to amplify and switch electronic signals; a semiconductor device with more than one positive-negative (pn) junction, which can either amplify current or voltage, or act as an on-off switch, a device incorporating semiconductor material and suitable contacts capable of performing electrical functions (such as voltage, current or power amplification) with low power requirements; an electronic switch that allows a (relatively) large amount of current to flow when a (relatively) small voltage is applied (just like a light switch can provide a large amount of electric energy to a lamp when a small amount of mechanical energy is expended); an electronic device that controls current flow without use of a vacuum; a regulator of current or voltage flow; an electronic device that can regulate electricity and act as an on/off switch; Diode-Transistor Logic (DTL, a class of digital circuits built from bipolar junction transistors (BJT), diodes and resistors).

In some embodiments, the cannula further comprises a handle having a radiation shielding PIG. The handle may be generally clear, translucent, transparent, pigmented, colored, or opaque. In some embodiments, the handle is constructed from a plastic, a glass (e.g., durable glass, e.g., Gorilla® Glass), or a combination thereof. In some embodiments, the handle is constructed from a material comprising a polyetherimide, poly (methyl methacrylate), acrylic polysulfone, polycarbonate, polypropylene, stainless steel, aluminum, or polyether ether ketone.

The present invention also features a PIG having an internal chamber, wherein the PIG comprises a sensor adapted for detecting a presence of a radioactive source or a carrier within the internal chamber. The sensor is operatively connected to both a power source and an alert system. Upon detection of the presence of a radioactive source or the carrier within the internal chamber the sensor triggers the alert system to notify a user that the radioactive source is within the internal chamber of the PIG.

In some embodiments, the sensor can detect the presence of a mass being stored within the internal chamber, the mass includes a radioactive source. In some embodiments, the sensor is an optical sensor. In some embodiments, the sensor is an electrical system. For example, in some embodiments, the sensor is a transistor (e.g., a solid-state transistor, a metal-oxide-semiconductor field-effect transistor (MOSFET), etc.). In some embodiments, the sensor is a non-electrical system (e.g., phosphorus). In some embodiments, the alert system provides a visual alert. In some embodiments, the alert system provides an audio alert.

In some embodiments, the PIG is generally clear, translucent, transparent, pigmented, colored, or opaque. In some embodiments, the PIG is constructed from a plastic, a glass (e.g., durable glass, e.g., Gorilla® Glass), or a combination thereof. In some embodiments, the PIG is constructed from a material comprising a polyetherimide, poly (methyl methacrylate), acrylic polysulfone, polycarbonate, polypropylene, stainless steel, aluminum, or polyether ether ketone.

The present invention also features a PIG having an internal chamber, wherein the PIG comprises a sensor adapted for detecting the removal of a radioactive source or the carrier within the internal chamber. The sensor is operatively connected to both a power source and an alert system. Upon detection of the removal of a radioactive source or the carrier within the internal chamber the sensor triggers the alert system to notify a user that the radioactive source is removed from the internal chamber of the PIG.

In some embodiments, the sensor can detect the presence of a radioactivity within the internal chamber. In some embodiments, the sensor can detect the presence of a mass being stored within the internal chamber, the mass includes a radioactive source.

In some embodiments, the sensor is an optical sensor. In some embodiments, the sensor is an electrical system. For example, in some embodiments, the sensor is a transistor (e.g., a solid-state transistor, a metal-oxide-semiconductor field-effect transistor (MOSFET), etc.). In some embodiments, the sensor is a non-electrical system (e.g., phosphorus). In some embodiments, the alert system provides a visual alert. In some embodiments, the alert system provides an audio alert.

In some embodiments, the PIG is generally clear, translucent, transparent, pigmented, colored, or opaque. In some embodiments, the PIG is constructed from a plastic, a glass (e.g., durable glass, e.g., Gorilla® Glass), or a combination thereof. In some embodiments, the PIG is constructed from a material comprising a polyetherimide, poly (methyl methacrylate), acrylic polysulfone, polycarbonate, polypropylene, stainless steel, aluminum, or polyether ether ketone.

The present invention also features methods of assembling a brachytherapy administering device. In some embodiments, the method comprises (a) obtaining a cannula subassembly comprising a generally hollow fixed shape cannula with a distal portion for placement around a portion of a globe of an eye, the distal portion has a radius of curvature between about 9 to 15 mm and an arc length between about 25 to 35 mm; a proximal portion having a radius of curvature between about an inner cross-sectional radius of the cannula and about 1 meter; an inflection point which is where the distal portion and the proximal portions connect with each other, the proximal portion is attached to a cap; (b) obtaining a handle subassembly comprising a handle having a first end and a second end, the first end is adapted to removably engage the cap of the cannula subassembly; a radiation shielding PIG for shielding radiation disposed in the handle; a channel disposed in the handle and the PIG, the channel aligns with the hollow fixed shape cannula when the first end of the handle engages the cap of the cannula subassembly; and an advancing means for advancing a brachytherapy system; (c) loading a brachytherapy system into the channel in the handle subassembly such that the brachytherapy system engages the advancing means, the brachytherapy system comprises a spiral cut tube having a first end and a second end and a radioactive brachytherapy source (RBS) disposed on the first end of the spiral cut tube, the brachytherapy system is inserted into the channel such that the RBS is oriented toward the first end of the handle of the handle subassembly; and (d) engaging the first end of the handle of the handle subassembly with the cap of the cannula subassembly.

In some embodiments, the handle subassembly further comprises an actuator connected to the advancing means, the actuator functions to manipulate the advancing means to control movement of the brachytherapy system. In some embodiments, the actuator is connected to the advancing means via a control cable system.

A locking means may secure the cannula subassembly and the handle subassembly together. In some embodiments, the locking means includes one or more screws. In some embodiments, the advancing means for advancing the brachytherapy system is a plunger mechanism. In some embodiments, the cannula assembly further comprises a lights system, the light system functions to emit light at a tip of the cannula. The light system may be attached to the handle by pressing the light system into a groove disposed on an outer surface of the handle. A light source may be engaged with the light system.

The present invention also features a brachytherapy administering device comprising (a) a cannula subassembly comprising a generally hollow fixed shape cannula with a distal portion for placement around a portion of a globe of an eye, the distal portion has a radius of curvature between about 9 to 15 mm and an arc length between about 25 to 35 mm; a proximal portion having a radius of curvature between about an inner cross-sectional radius of the cannula and about 1 meter; an inflection point which is where the distal portion and the proximal portions connect with each other, the proximal portion is attached to a cap; and (b) a handle subassembly comprising a handle having a first end and a second end, the first end is adapted to removably engage the cap of the cannula subassembly; a radiation shielding PIG for shielding radiation disposed in the handle; a channel disposed in the handle and the PIG, the channel is positioned such that the channel aligns with the hollow fixed shape cannula when the first end of the handle engages the cap of the cannula subassembly; and an advancing means for advancing a RBS.

In some embodiments, the handle subassembly further comprises an actuator connected to the advancing means, the actuator functions to manipulate the advancing means. In some embodiments, the actuator is connected to the advancing means via a control cable system.

In some embodiments, the device further comprises a locking means for securing the cannula subassembly and the handle subassembly together. In some embodiments, the locking means includes one or more screws. In some embodiments, the advancing means for advancing a RBS is a plunger mechanism. In some embodiments, the cannula assembly further comprises a light system, the light system functions to emit light at a tip of the cannula. A groove may be disposed in the handle, wherein the groove is adapted to snugly wrap around the light system to connect the light system of the cannula subassembly to the handle subassembly. In some embodiments, the device further comprises a light source for engaging with the light system.

### BRIEF DESCRIPTION OF THE DRAWINGS

FIG. 1A is a schematic cross sectional view of an eye wherein a cannula is positioned around the eye (between the Tenon's capsule and sclera).

FIG. 1B is an exploded view of FIG. 1A. A light source is disposed at the tip of the cannula.

FIG. 2 is a side view of an embodiment of the cannula of the present invention. An actuator handle is connected to an advancing means.

FIG. 3A is a first side view of an embodiment of the system of the present invention.

FIG. 3B is a second side view of the cannula, pig, and handle of FIG. 3A.

FIG. 3C is a third side view of the cannula of FIG. 3A, wherein the RBS and spiral cut tube can be visualized through the PIG (and handle).

FIG. 3D is a front view of the cannula of FIG. 3A. A channel is disposed in the handle/PIG allowing the fiber of the light system to be secured to the handle/PIG.

FIG. 3E is a front view of the cannula of FIG. 3A. A channel is disposed in the handle/PIG allowing the fiber of the light system to be secured to the handle/PIG.

FIG. 4A is a side view of a brachytherapy system comprising a RBS attached to a spiral cut tube. A solid shaft is disposed on the end of the spiral cut tube opposite the RBS. The RBS may be generally cylindrical or spherical.

FIG. 4B is a side view of a brachytherapy system comprising a RBS attached to a spiral cut tube. A solid shaft is disposed on the end of the spiral cut tube opposite the RBS. The RBS is generally cylindrical.

FIG. 4C is a side view of a brachytherapy system comprising a RBS attached to a spiral cut tube. A solid shaft is disposed on the end of the spiral cut tube opposite the RBS. The RBS is generally disc-shaped.

FIG. 5 is a side in-use view of an embodiment of the cannula of the present invention.

FIG. 6 is back in-use view of the cannula of FIG. 5.

FIG. 7 is a side view of an actuator handle connected to the handle.

FIG. 8A is a perspective view of a light used to engage the light connector component.

FIG. 8B is a side cross sectional view of the light connector component.

FIG. 9 is a side view of a cannula of the present invention comprising a secondary radiation shield disposed on the end of the handle.

FIG. 10 is an exploded view of the cannula of the present invention, wherein the cannula is being assembled.

FIG. 11 is a perspective view of a radiation shield.

FIG. 12A is a side view of an embodiment of the present invention comprising a first sensor disposed at the tip of the cannula and a second sensor disposed in the PIG/handle.

FIG. 12B is a side view of the device of FIG. 12A, wherein the RBS is positioned in the PIG/handle.

FIG. 12C is a schematic view of an alert device/monitor, wherein the monitor is configured to calculate radiation at the tip and in the PIG/handle via the sensors.

FIG. 12C shows that radiation is detected in the PIG/handle.

FIG. 12D is a side view of the device of FIG. 12A, wherein the RBS is positioned at the tip of the cannula (e.g., at a treatment zone).

FIG. 12E is a schematic view of an alert device/monitor, wherein the monitor is configured to calculate radiation at the tip and in the PIG/handle via the sensors. FIG. 12C shows that radiation is detected at the tip of the cannula.

FIG. 13 is a schematic representation of the sensor's calculation of treatment time (e.g., time the target is exposed to the RBS).

FIG. 14 is a side view of the device of the present invention comprising a window for visualization of the seed and/or spiral cut tube and/or advancing means, or the like (e.g., the channel that the seed and spiral cut tube pass through in the PIG).

FIG. 15A is a side view of a brachytherapy system comprising a RBS attached to a spiral cut tube and a solid shaft disposed on the end of the spiral cut tube opposite the RBS. A marker is disposed on the solid shaft.

FIG. 15B is a side view of a brachytherapy system comprising a RBS attached to a spiral cut tube and a solid shaft disposed on the end of the spiral cut tube opposite the RBS. A marker is disposed on the RBS.

FIG. 15C is a side view of a brachytherapy system comprising a RBS attached to a spiral cut tube and a solid shaft disposed on the end of the spiral cut tube opposite the RBS. A marker is disposed on the solid spiral cut tube.

FIG. 16 is a side view of the device of FIG. 14 comprising a landmark (secondary marker) disposed on the PIG.

FIG. 17 is a side view of a device of the present invention featuring a radiation optic switch (switch sensor) for detecting the presence of the RBS in a treatment zone.

### DESCRIPTION OF PREFERRED EMBODIMENTS

### Cannula

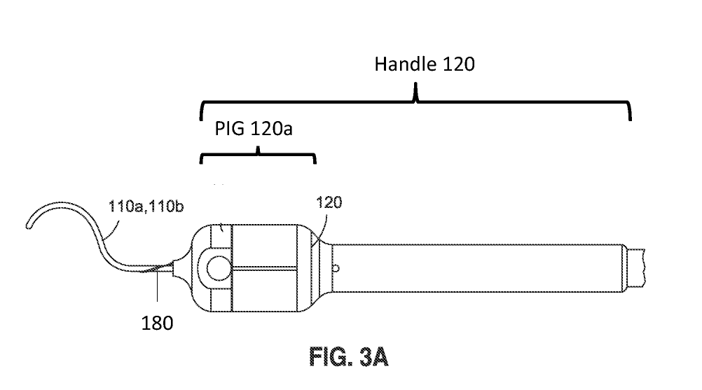

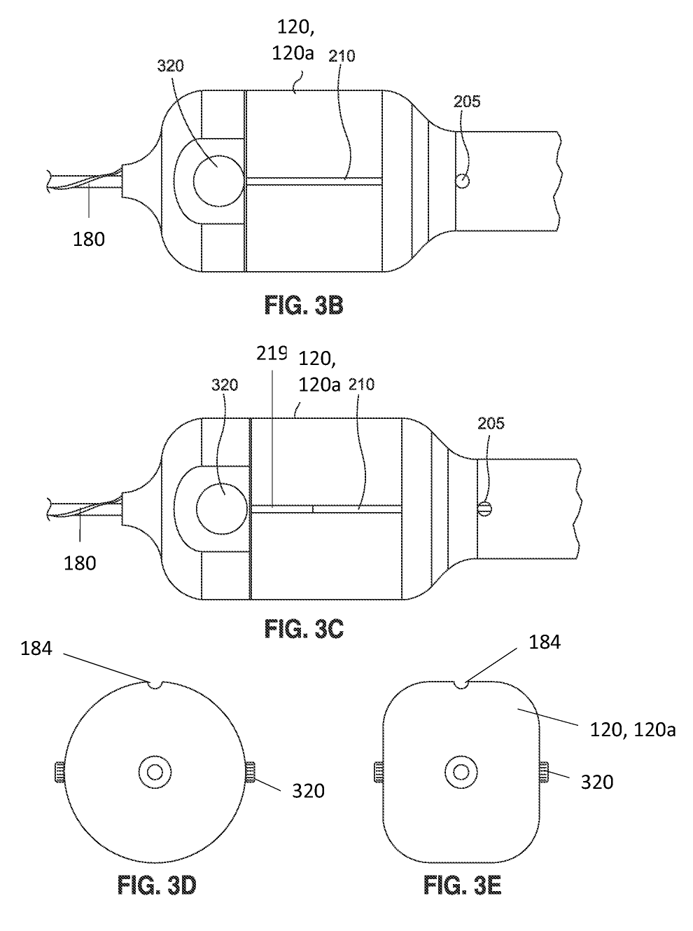

The present invention features a cannula comprising a distal portion 110a for placement around a portion of a globe of an eye; a proximal portion 110b; an inflection point, which is where the distal portion and the proximal portions connect with each other. The cannula may be divided into a first assembly (e.g., cannula subassembly 124) and a second assembly (e.g., handle subassembly 125), the cannula subassembly 124 comprising the cannula distal portion 110a and proximal portion 110b and the handle subassembly 125 comprising the handle 120 (e.g., with radioactive shielding PIG 120a) (e.g., see FIG. 10). As shown in FIG. 3A, the PIG 120a may be part of the handle 120.

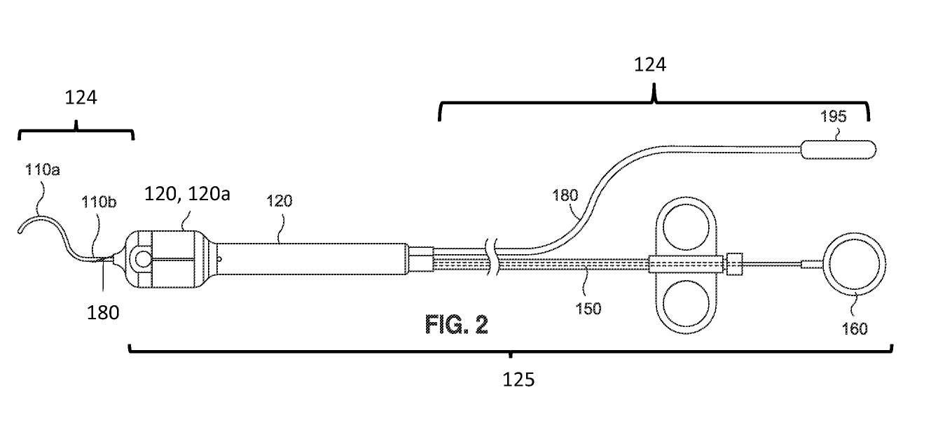

As shown in FIG. 2, in some embodiments, the cannula subassembly 124 further comprises a light system (e.g., light fiber 180) and a light connector component 195, wherein the connector component is adapted to engage a light source 199. In some embodiments, the handle subassembly 125 further comprises a control cable system 150 (with an actuator handle 160). The light fiber 180 may temporarily be secured to the handle 120 by inserting the light fiber 180 into a light fiber channel 184 disposed in the handle 120, for example on the outer surface of the handle 120 (see FIG. 3D, FIG. 3E).

The first assembly (e.g., cannula subassembly 124) may attach to the second assembly (e.g., handle subassembly) via an attachment means. For example, the proximal portion 110b of the cannula may be connected to the handle subassembly 125 via an attachment means, for example a hub or connector component 310 disposed on the proximal portion 110b (see FIG. 10) that engages the outer end of the handle 120 (handle subassembly 125). In some embodiments, the connector component 310 engages the outer end of the handle 120 and is secured to the handle 120 via a securing mechanism. In some embodiments, the securing mechanism includes a screw and aperture mechanism. For example, one or more first apertures (e.g., threaded apertures) are disposed in the connector component 310 and one or more second apertures (e.g., threaded apertures) are disposed in the handle 120 (e.g., the outer end). The first apertures are positioned to align with the second apertures when the connector component 310 is attached to the handle 120. The first and second apertures (e.g., threaded apertures) are adapted to receive thumb screws 320 (for securing the connector component 310 to the handle 120). The connector component 310 can be slid onto the handle 120 and the first apertures are aligned with the second apertures. The thumb screws 320 can be driven through the apertures to secure the first connector component 310 and handle 120 together.

An RBS (e.g., RBS 220 disposed on an end of a spiral cut tube 210, for example) can be loaded into the device. For example, the RBS/spiral cut tube 210 is loaded into a channel 219 disposed in the handle/PIG 120, wherein the RBS/spiral cut tube 210 can engage an advancing means (e.g., a plunger, etc., for advancing the RBS/spiral cut tube 210 from the handle/PIG 120 to the distal portion/proximal portion of the cannula). In some embodiments, the RBS/spiral cut tube 210 is temporarily held in a seed-loading dummy (e.g., radiation shield device) while the RBS/spiral cut tube 210 is secured within the device (e.g., the channel 219 in the handle/PIG 120). For example, a solid shaft 210a may be disposed on the end of the spiral cut tube 210, which engages the advancing means (e.g., plunger). The RBS 220 may be shielded by the seed-loading dummy while the solid shaft 210a is engaged and secured to the plunger, e.g., by tightening screws. The tightening screws may be accessible via tightening screw apertures 205 disposed in the handle/PIG 120. In some embodiments, the seed-loading dummy has a channel adapted to hold the RBS/spiral cut tube 221. The channel can be aligned with the channel 219 in the handle/PIG 120 so that the RBS/spiral cut tube 210 can be easily transferred from the seed-loading dummy to the device.

### Handle/Radioactive Shielding Pig (Ultem® 1000)

The handle 120 is attached to the proximal portion of the cannula (e.g., fixedly attached, removably attached—e.g., via an attachment means, etc.). The handle 120 comprises a radioactive shielding PIG 120a. In some embodiments, at least a portion of the handle 120 and/or PIG 120a may be generally clear, translucent, or transparent. As used herein, the terms “clear,” “translucent,” and “transparent” refer to a property of a material that allows visualization of light, an object, or a shadow.

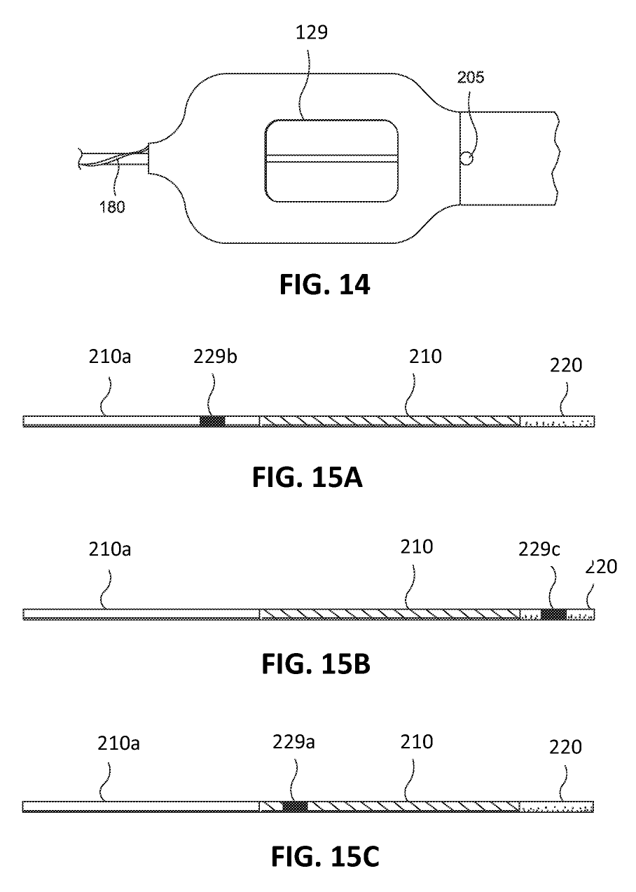

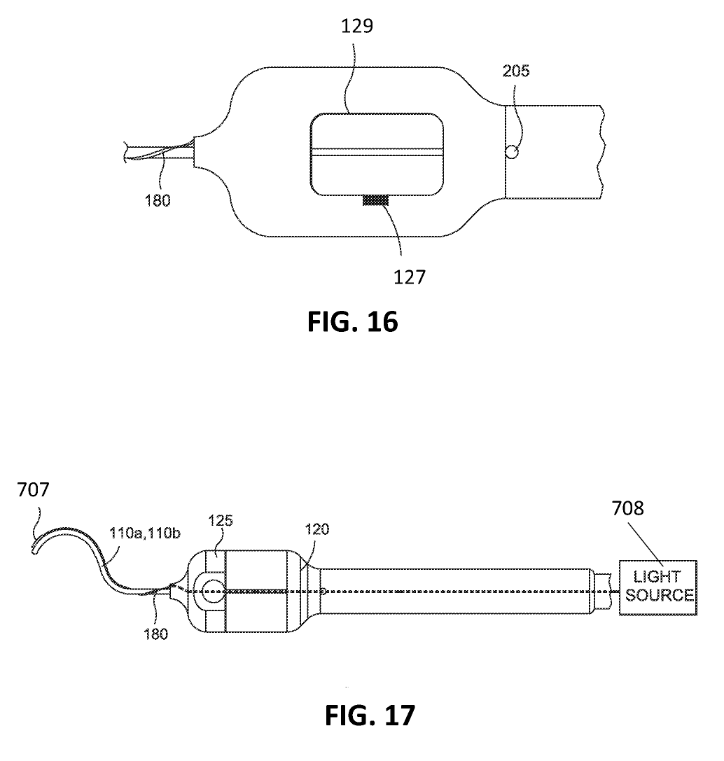

The PIG 120a may alternatively be constructed from a material that is not clear, translucent, or transparent (e.g., a metal, etc.) if a window (or aperture) is disposed in the PIG 120a allowing visualization of the seed and/or spiral cut tube and/or advancing means (e.g., plunger), or the like. For example, FIG. 14 and FIG. 16 shows a window 129 disposed in the PIG 120a allowing visualization of the seed and/or spiral cut tube and/or advancing means, or the like (e.g., the channel that the seed and spiral cut tube pass through in the PIG 120a).

The handle 120 and/or PIG 120a may be constructed from a material comprising plastic, glass (e.g., Gorilla® Glass), the like, or a combination thereof.

In some embodiments, the handle 120 (e.g., the radioactive shielding PIG 120a) is constructed from a material comprising a polyetherimide material (e.g., Ultem® 1000), an acrylic, or a combination thereof. Alternatively, in some embodiments, the handle 120 is constructed from a material comprising acrylic, poly(methyl methacrylate) (PMMA), polysulfone, polycarbonate, polypropylene, the like, or a combination thereof. The present invention is not limited to translucent, transparent, and/or clear materials, nor is the present invention limited to the aforementioned materials. For example, in some embodiments, the handle 120 is constructed from a material comprising a stainless steel, aluminium, titanium, elgiloy, lead, the like, or a combination thereof. For example, if sensors were incorporated into the tip and/or handle/PIG 120, visual detection of the position of the RBS within the cannula or handle/PIG 120 may not be necessary. Generally, the handle/PIG 120 is constructed from lightweight material(s), which can be more comfortable for a surgeon or physician to use (e.g., the handle/PIG is lighter than a handle/PIG 120 made from lead, for example).

The PIG is of sufficient thickness so as to block radiation (e.g., beta radiation), to block bremsstrahlung radiation, and to in some manner allow visualization of the seed and/or spiral cut tube and/or the plunger.

The polyetherimide material (e.g., Ultem® 1000) may be radiation-resistant, durable, and translucent/transparent. The polyetherimide material (e.g., Ultem® 1000) may provide shielding to protect a physician prior during and after use of the cannula.

In some embodiments, the PIG 120a is positioned at or near a first end of the handle 120 (e.g. the end that attaches to the proximal portion 110b of the cannula). In some embodiments, the PIG 120a is positioned at or near a middle portion of the handle 120. In some embodiments, the PIG 120a is positioned at or near a second end of the handle 120. In some embodiments, the PIG 120a is positioned in between the first end and the middle portion of the handle 120. In some embodiments, the PIG 120a is positioned in between the second end and the middle portion of the handle 120.

In some embodiments, the handle 120 (e.g., the radioactive shielding PIG 120a) is generally cylindrical, oval, octagonal, rectangular, or irregular in shape. For example, FIG. 3D shows a generally cylindrical handle/PIG 120. FIG. 3E shows a handle/PIG 120 having at least one flat edge. A visual landmark 127 (e.g., marking, etc.) may be disposed on the handle/PIG 120, functioning as a reference point for orientation of the device, for example during a surgical procedure. In some embodiments, the thumbscrews 320 function as the visual landmark. In some embodiments, the channel 184 holding the light fiber 180 functions as the visual landmark. In some embodiments, a flat edge of the handle/PIG 120 functions as a visual landmark. In some embodiments, the visual landmark is a marking, a protrusion, or an indentation disposed on the handle/PIG. The visual landmark is not limited to the aforementioned examples.

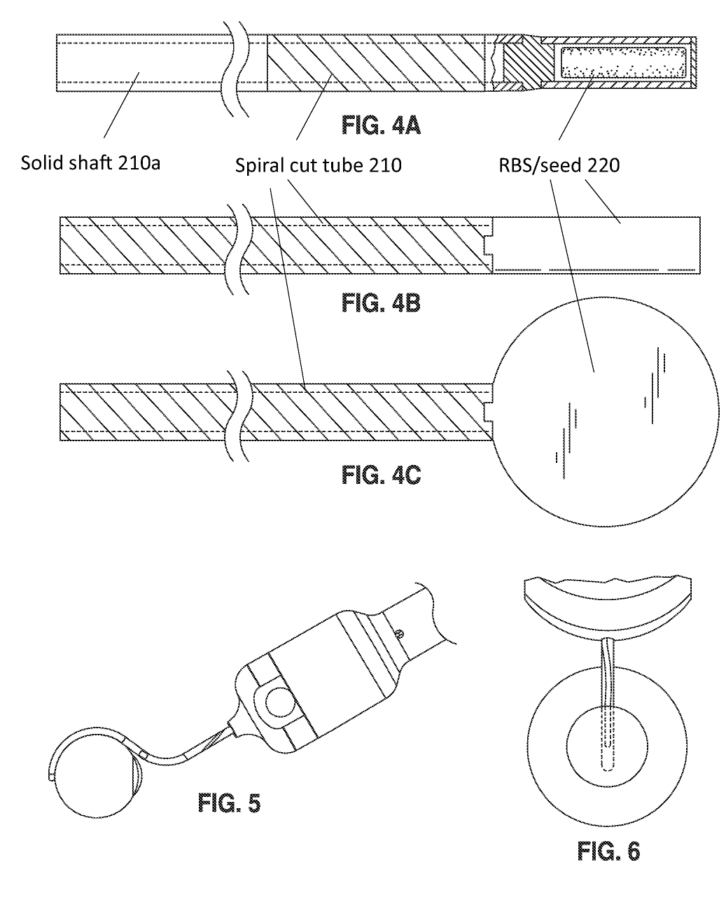

The handle 120 (e.g., the radioactive shielding PIG 120a) may be constructed in a variety of sizes. For example, in some embodiments, the handle 120 (e.g., the radioactive shielding PIG 120a) has an outer diameter between about 2.0 and 3.0 cm. In some embodiments, the handle 120 (e.g., the radioactive shielding PIG 120a) has an inner diameter of less than or equal to about 0.1 cm. The inner and/or outer diameters of the handle 120 (e.g., the radioactive shielding PIG 120a) may allow a physician to visualize a position of a radionuclide brachytherapy source (RBS) (e.g., a radioactive seed 220) prior to deployment and after retrieval. The translucency/transparency of the handle 120 (e.g., radioactive shielding PIG 120a) may allow a physician to visualize a position of a radioactive seed (RBS) 220 prior to deployment and after retrieval (see FIG. 3A, FIG. 4A).

In addition to allowing the physician to visualize a position of the radioactive seed (RBS 220) prior to deployment and after retrieval, the polymer may also provide for the required shielding to protect the physician prior, during and after the procedure, while the diameter allows the physician to visualize the seed (RBS 220) in position in the PIG 120a prior to deployment. For example, when the actuating handle has pushed the advancing means as far forward as possible and the seed has reached the target zone, the channel 219 in the handle/PIG 120 will be filled with the spiral cut tube 210 (e.g., optionally a portion of the solid shaft). When the RBS 220 is withdrawn via the actuator handle and advancing means, the spiral cut tube 210 (e.g., RBS 220) may be visible in the handle/PIG (see FIG. 3C). The channel 219 may be translucent.

In some embodiments, the clear, translucent, and/or transparent handle/PIG 120 is advantageous in that it allows a physician to visualize and confirm the fully deployed RBS by noting the extent of travel and position of the advanced spiral cut tube 210.

In some embodiments, a polyetherimide cylinder having an outside diameter of 2.3 cm and a length of 2.2 cm containing a hole drilled through the symmetry axis of diameter ≦0.1 cm will provide shielding such that when using a 10 mCi Sr-90/Y-90 source, a hand dose of ≦0.01 mSv will be received if the device is held for 10 minutes at the point of shielding.

### Spiral Cut Tube And Radioactive Brachytherapy Source (RBS)

The present invention also features a spiral cut tube 210 wherein a RBS/seed 220 is disposed on an end (e.g., the first end) of the flexible spiral cut tube 210. The flexible spiral cut tube 210 may be used for advancing a RBS (e.g., radionuclide brachytherapy source) 220. The spiral cut tube 210 can move through the cannula and handle/PIG 120 (e.g., in a channel 219 within the handle/PIG 120) for delivering the RBS 220 to the tip of the cannula. The RBS may be secured to the end of the spiral cut tube 210 via a securing means (e.g., welding, etc.).

In some embodiments, the spiral cut tube has a cut angle of about 5.12 degrees. In some embodiments, the spiral cut tube has a cut angle between about 4 to 4.5 degrees. In some embodiments, the spiral cut tube has a cut angle between about 4.5 to 5 degrees. In some embodiments, the spiral cut tube has a cut angle between about 5 to 5.5 degrees. In some embodiments, the spiral cut tube has a cut angle between about 5.5 to 6 degrees. In some embodiments, the spiral cut tube has a cut angle between about 6 to 6.5 degrees. In some embodiments, the spiral cut tube has a cut angle less than about 4 degrees. In some embodiments, the spiral cut tube has a cut angle more than about 6.5 degrees.

In some embodiments, the spiral cut tube is about 2.3 inches in length as measured from the first end to the second end. In some embodiments, the spiral cut tube is between about 1 to 2 inches in length as measured from the first end to the second end. In some embodiments, the spiral cut tube is between about 2 to 3 inches in length as measured from the first end to the second end. In some embodiments, the spiral cut tube is between about 3 to 4 inches in length. In some embodiments, the spiral cut tube is less than about 1 inch in length. In some embodiments, the spiral cut tube is more than about 4 inches in length.

In some embodiments, the cuts on the spiral cut tube are about 0.02 inches apart. In some embodiments, the cuts on the spiral cut tube are between about 0.005 to 0.01 inches apart. In some embodiments, the cuts on the spiral cut tube are between about 0.01 to 0.02 inches apart. In some embodiments, the cuts on the spiral cut tube are between about 0.02 to 0.03 inches apart. In some embodiments, the cuts on the spiral cut tube are less than about 0.005 inches apart. In some embodiments, the cuts on the spiral cut tube are more than about 0.03 inches apart.

In some embodiments, the cuts on the spiral cut tube are about twenty thousandth of an inch apart. In some embodiments, the cuts on the spiral cut tube are between about five thousandth and ten thousandth of an inch apart. In some embodiments, the cuts on the spiral cut tube are between about ten thousandth and twenty thousandth of an inch apart. In some embodiments, the cuts on the spiral cut tube are between about twenty thousandth and thirty thousandth of an inch apart. In some embodiments, the cuts on the spiral cut tube are less than about five thousandth of an inch apart. In some embodiments, the cuts on the spiral cut tube are more than about thirty thousandth of an inch apart.

In some embodiments, the cuts on the spiral cut tube are about 0.001 inches in width. In some embodiments, the cuts on the spiral cut tube are between about 0.0001 to 0.001 inches in width. In some embodiments, the cuts on the spiral cut tube are between about 0.001 to 0.01 inches in width. In some embodiments, the cuts on the spiral cut tube are less than about 0.0001 inches in width. In some embodiments, the cuts on the spiral cut tube are more than about 0.001 inches in width.

In some embodiments, the cuts on the spiral cut tube are about one thousandth of an inch in width. In some embodiments, the cuts on the spiral cut tube are between about one ten-thousandth and one thousandth of an inch in width. In some embodiments, the cuts on the spiral cut tube are between about one thousandths and ten thousandth of an inch in width. In some embodiments, the cuts on the spiral cut tube are less than about one ten-thousandth of an inch in width. In some embodiments, the cuts on the spiral cut tube are more than about ten thousandth of an inch in width.

In some embodiments, a solid shaft 210a is disposed on the opposite end (e.g., second end) of the spiral cut tube 210. The solid shaft 210a may engage the advancing means (e.g., plunger) to secure the spiral cut tube 210 and RBS 220 to the advancing means.

As shown in FIG. 4A-4C, the RBS 220 may be constructed in a variety of shapes and sizes including but not limited to a generally cylindrical shape, a generally oval shape, a generally disc shape, a generally annulus shape, a generally spherical shape, an irregular shape, the like, or a combination thereof.

In some embodiments, the RBS is a floating radioactive seed 220. The floating radioactive seed 220 may float between two fixed points in the flexible spiral cut tube 210. The spiral cut tube 210 may draw the seed 220 along by friction and the restrictions of the fixed endpoints. The spiral cut tube 210 may not have a direct push or pull rod, which may help eliminate longitudinal compressive forces put on a floating radioactive seed 220 during deployment and may help eliminate elongation forces during withdrawal. Free space around the floating radioactive seed 220 may be considered a safety zone of space ensuring that the seed 220 is not compressed.

The actuator handle 160 can advance the spiral cut tube 210 and floating radioactive seed 220 (e.g., via the advancing means) out of the handle 120. When it encounters curves in the cannula (e.g., distal portion 110a, proximal portion 110b), the spiral cut tube 210 flexes. The spiral cut tube 210 becomes a straight tube in the straight portions of the cannula and flexes during curved portions of the cannula.

In some embodiments, as shown in FIG. 15C, a visual marker (e.g., first visual marker 229a) is disposed on the spiral cut tube 210. In some embodiments, as shown in FIG. 15A, a visual marker (e.g., second visual marker 229b) is disposed on the solid shaft 210a. In some embodiments, as shown in FIG. 15B, a visual marker (e.g., third visual marker 229c) is disposed on the RBS 220. In some embodiments, the visual marker 229 on the spiral cut tube 210 and/or solid shaft 210a and/or RBS 220 is visible from outside the handle or the cannula. For example, a window 129 or aperture may be disposed in the PIG/handle, which allows the markers to be visualized from outside the handle or cannula.

In some embodiments, a visual marker may be disposed on the control cable system 150. In some embodiments, a visual marker may be disposed on the control cable system 150 at where it intersects with the solid shaft 210a.

### Light System

The present invention may further comprise a light system for illumination purposes, for example for illuminating a posterior portion of the eye, a portion of the subtenon space, landmarks (e.g., macula lutea, fovea centralis, optic disk, etc.), and the like. The light system (e.g., fiber 180) comprises a tip 260 (e.g., a terminating end). The tip 260 of the light system (e.g., fiber 180) may be positioned at or near a portion of the intended position of the radioactive seed 220 (e.g., the treatment position). The light tip 260 may be positioned to be at the middle portion of the radioactive seed 220 when the seed 220 is in the intended treatment position. Light emitted from the tip 260 may be directed into the vitreous cavity. The light emitted from the tip 260 may be used to indicate the middle portion of the radioactive seed 220.

The light emitted from the light system (e.g., fiber 180) may be directed at an angle. For example, in some embodiments, the tip 260 is cut at an angle. For example, in some embodiments, the tip 260 is cut at an angle between about 40 to 50 degrees. In some embodiments, the tip 260 is cut at an angle between about 50 to 60 degrees. In some embodiments, the tip 260 is cut at an angle between about 60 to 70 degrees. In some embodiments, the tip 260 is cut at an angle between about 70 to 75 degrees.

The angle of the tip 260 angles the light emitted from the light system (e.g., fiber 180). Alternatively, in some embodiments, a lens is used to angle the light emitted (e.g., similar to lens found on arthroscope, a sapphire lens, etc.). In some embodiments, a reflective component (e.g., a mirror) is used to angle the light emitted.

The light system may comprise one or more fibers 180, for example a group of three fibers (e.g., fiber cable). The fiber(s) may be constructed from a material comprising poly(methyl methacrylate) (PMMA), glass, the like, or a combination thereof. The fibers are not limited to the aforementioned materials. The fibers 180 may run along the outside of the cannula (e.g., outside the distal portion, proximal portion). In some embodiments, the fibers in combination with the cannula (e.g., the proximal and distal portions) are covered with a polyethylene terephthalate (PET) heat shrink tube. In some embodiments, the fibers 180 are tacked to the cannula with an adhesive (e.g. UV adhesive). The fiber(s) 180 may be secured to the handle/PIG by inserting the fiber(s) into a channel disposed on the handle/PIG (see FIG. 3D, FIG. 3E).

The cannula (e.g., the proximal portion 110b and distal portion 110a) may be covered with a PET heat shrink tube without the fibers. With or without the fibers, the cannula may be covered with a polyether ether ketone (PEEK) heat shrink tube.

### Illumination Connection/Light Connecting Component

The present invention may features a light connecting component 195 (e.g., light source adaptor) disposed on the end of the light system (e.g. fiber 180). The light connecting component 195 (e.g., light source adaptor) is adapted to engage a light source 199 (see FIG. 8A). The light source 199 may include but is not limited to a battery powered light source (e.g., Scintillant® Surgical Light). In some embodiments, an o-ring system (e.g., one, two, three, or more than three o-rings 185) is disposed in the light connecting component 195 (see FIG. 8B), which help secure the light source 199 to the light connecting component 195.

The light connecting component 195 may be constructed from a variety of materials, for example polycarbonate (e.g., black polycarbonate or clear polycarbonate), polyetherimide (e.g., Ultem® 1000), polyoxymethylene, light-blocking pigmented polymers, metals, ceramics, aluminum, stainless steel, the like, or a combination thereof. The o-rings 185 may be constructed from a material comprising silicone, Buna-N, latex, ethylene-propylene, polyurethane, neoprene, fluorocarbon, fluorosilicone, the like, or a combination thereof.

Alternatively to o-ring system, a lay& of polymer may line the inside diameter of the light connecting component 195 to achieve an uninterrupted frictional fit over the length of the light connecting component 195. This can hold and lock the device in place at any given distance along the light source focal point within the light connecting component 195 with respect to the light fibers. The inside diameter may be textured to increase frictional fit over the length of the light connecting component 195 and lock the light source in place.

The light can be dimmed by defocusing/withdrawing the light source 199 (e.g., Scintillant® Surgical Light) away from the light fibers in the light connecting component 195 to reduce the light output to the target zone, while the device is continued to be held in place by the friction of the o-rings 185.

### Control Cable System And Actuator Handle

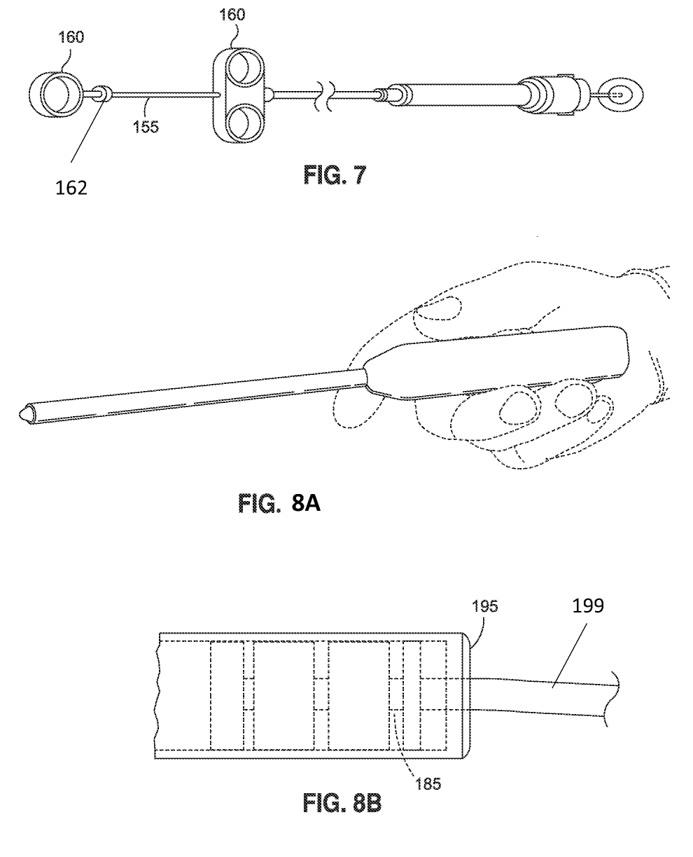

As shown in FIG. 2 and FIG. 7, a control cable system 150 with an actuator handle 160 may be used to deploy and retrieve the radioactive seed 220 (via an advancing means such as a plunger). The actuator handle 160 and control cable system can 150 advance the spiral cut tube 210 and radioactive seed 220 (e.g., RBS) out of the handle/PIG 120 and retract the spiral cut tube 210 and radioactive seed 220 (e.g., RBS) back into the handle 120. The control cable system 150 has a first end for connecting to the. advancing means (e.g., plunger), the advancing means engages the spiral cut tube 210 and/or the solid shaft 210a attached to the spiral cut tube 210. The advancing means (e.g., means of moving the RBS 220) is adapted to move the RBS/spiral cut tube from the handle/PIG 120 to the tip of the cannula and back.

The second end of the control cable system 150 is connected to the actuator handle 160. The actuator handle 160 features a stop collar 162, designed to allow for adjustment of the control cable system 150 and advancing means (e.g. plunger). For example, the stop collar 162 can be adjusted, thereby adjusting the overall placement of the RBS/spiral cut tube. This can allow for fine-tuning of the placement of the RBS 220 at the treatment zone at the tip of the cannula. In some embodiments, the adjustment of the stop-collar 162 position can be achieved with an Allen wrench.

The control cable system 150 does not interfere with positioning of the cannula. The control cable system 150 may comprise a central wire rope (e.g., constructed from a material comprising stainless steel coated with nylon, an elastic or super elastic material, Nitinol, elgiloy, combination rope, the like, or a combination thereof) surrounded by an outer tube (e.g., constructed from a material comprising polyvinyl chloride (PVC) with the inner diameter being lined with FEP Teflon, polytetrafluoroethylene (PTFE), fluorinated ethylene propylene (FEP), acrylic, the like, or a combination thereof).

In some embodiments, a portion of the control cable system 150, for example an upper portion 155, which is at or near the actuator handle 160, comprises a stainless steel tube. The stainless steel tube may help provide strength to the control cable system 150.

In some embodiments, a visual marker is disposed on the advancing means (e.g., the plunger). The visual marker functions as a reference point for determining the position of the advancing means (e.g., the plunger) and RBS/spiral cut tube 210, 220. In some embodiments, the PIG/handle 120 is constructed to allow for visualization of the visual marker on the advancing means (e.g., the plunger) (from outside the device), for example the PIG/handle 120 is generally clear, translucent, or transparent, or a window 127 (or aperture) is disposed in the PIG/handle (e.g., if the PIG/handle is not clear, translucent, or transparent). In some embodiments, an aperture is disposed in the PIG/handle 120, wherein the aperture is positioned such that when the advancing means is positioned in a treatment position the visual marker on the advancing means is visible through the aperture. In some embodiments, a secondary marker or landmark 127 is disposed in the PIG/handle 120 (e.g., see FIG. 16), wherein when the visual marker on the advancing means or the visual marker 229 of the spiral cut tube, RBS, or solid shaft is aligned with the secondary marker or landmark 127 on the PIG/handle 120, the RBS/spiral cut tube 210, 220 may be fully deployed (e.g., the RBS 220 may be at a treatment position).

In some embodiments, only the bulging portion of the PIG/handle is clear, translucent, or transparent. In some embodiments, only the slender portion of the PIG/handle is clear, translucent, or transparent. In some embodiment, the bulging portion is the PIG. In some embodiments, the slender (long tube-like portion) is the handle.

### Optional Secondary Radiation Shield

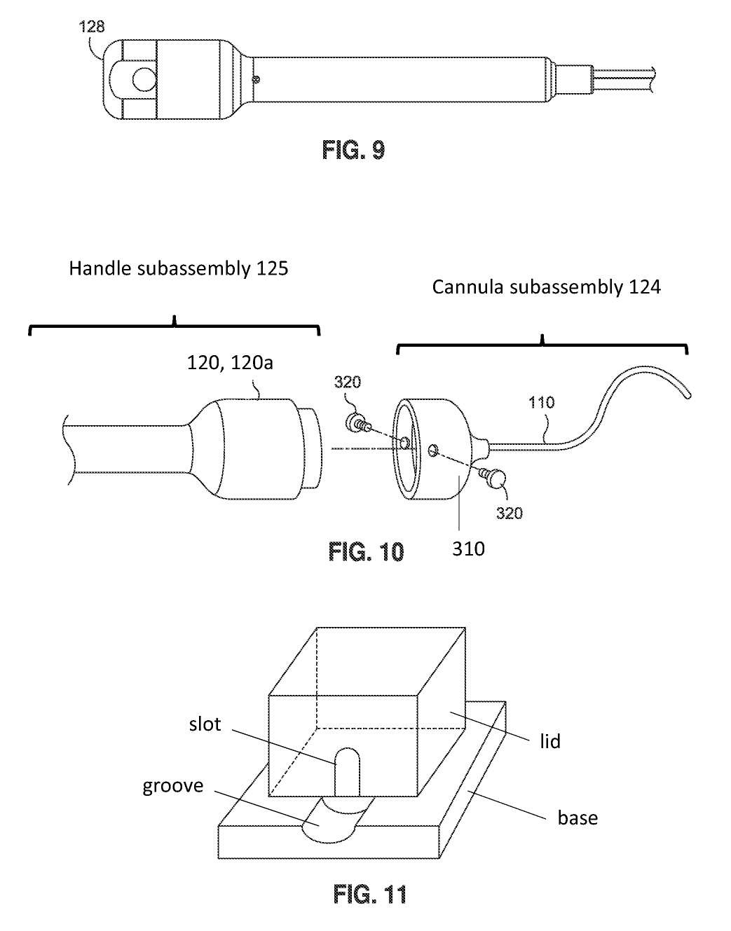

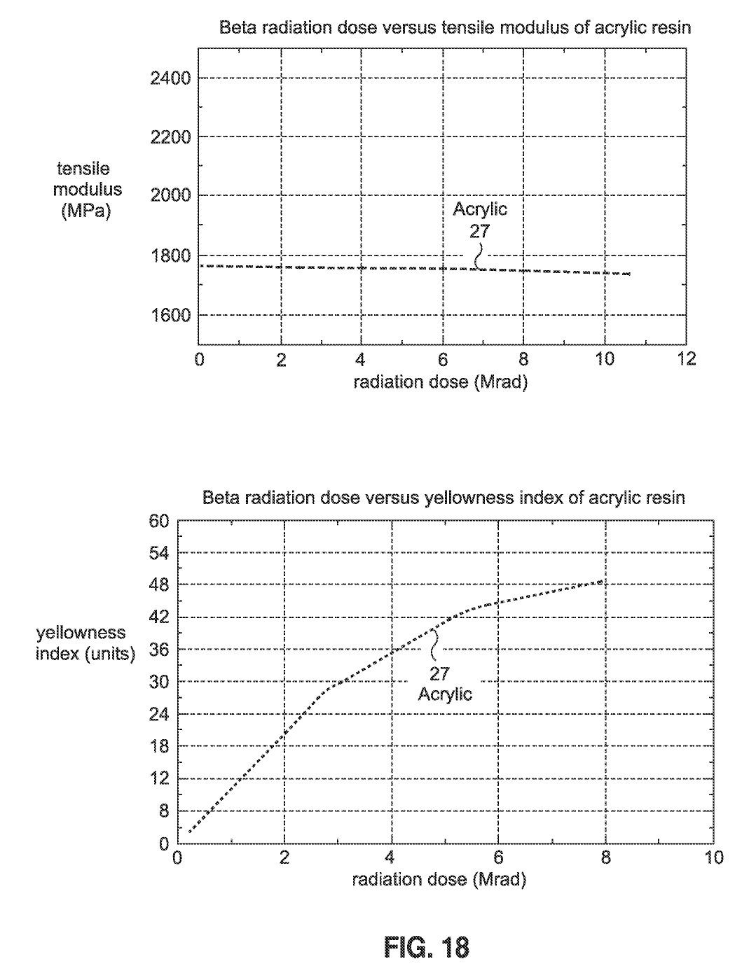

As shown in FIG. 9, in some embodiments, the present invention may further comprise a secondary radiation shield 128 for attaching to the handle 120 (e.g.; radioactive shielding PIG 120a) (see FIG. 17). The secondary radiation shield 128 may be constructed from a material comprising a polyetherimide material (e.g., Ultem® 1000). In some embodiments, the secondary radiation shield 128 attaches to the outer end of the handle 120 in the same manner in which the cannula subassembly 124 (e.g., connector component 310) attaches to the outer end of the handle 120.

### Sensors

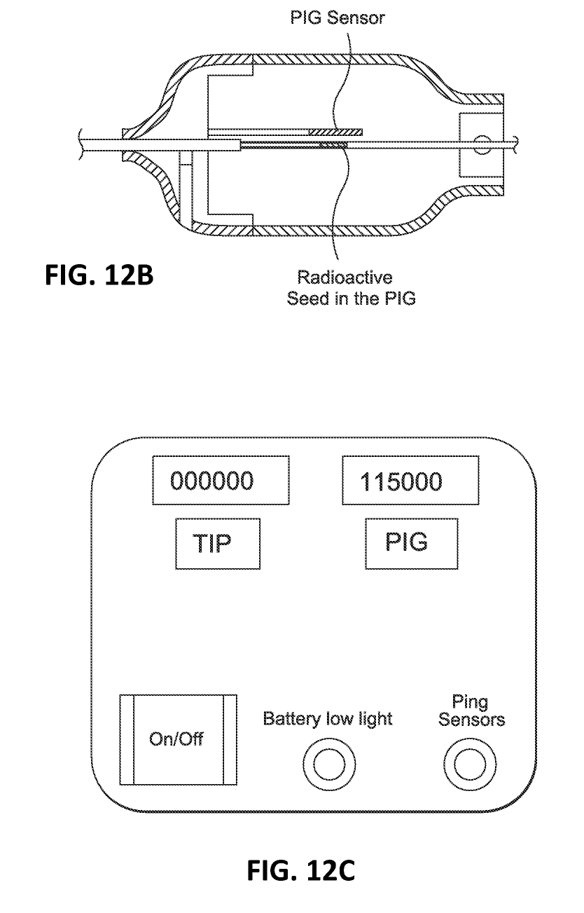

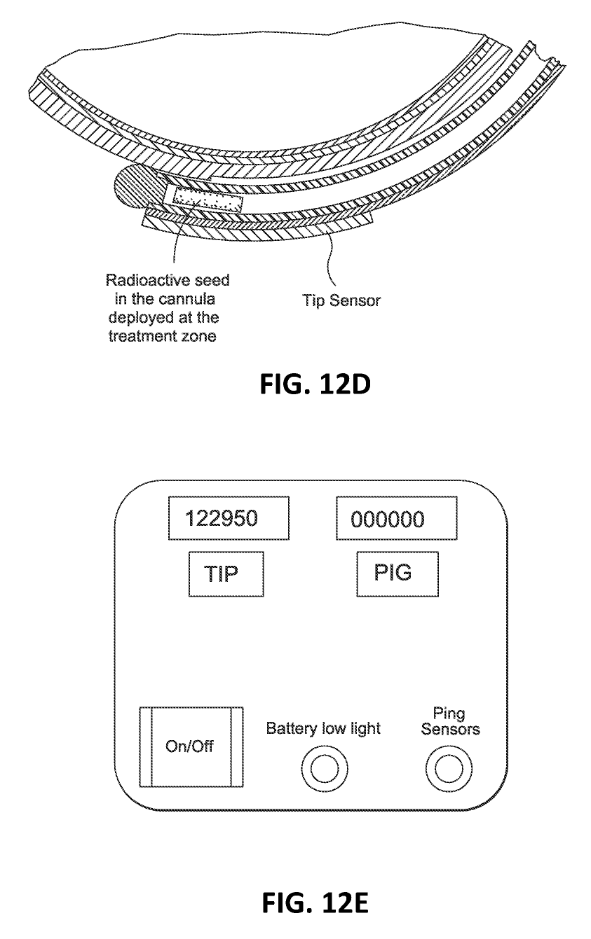

In some embodiments, the cannula further comprises a sensor system for detecting when the RBS 220 is advanced (e.g., see FIG. 12A-12E). For example, a device may comprise a sensor adapted to detect the presence of an RBS 220 at a position within the cannula (e.g., a treatment position), e.g., the sensor may be disposed at or near the tip of the cannula. Upon detection of the presence of the RBS at the position (e.g., treatment position) within the cannula the sensor triggers an alert system to notify a user that the RBS 220 is at the position within the cannula. In some embodiments, the sensor activates a light source when the RBS 220 is detected in the treatment zone. The sensor detecting the RBS 220 at the treatment zone can be used to confirm that the radiation is actually being emitted at the intended location (e.g., treatment zone).

In some embodiments, the PIG 120a may comprise a sensor adapted to detect the presence of an RBS 220 (or carrier) within its internal chamber or channel. Upon detection of the presence of the RBS 220 (or carrier) within the internal chamber or channel the sensor triggers an alert system to notify a user that the RBS 220 (or carrier) is within the internal chamber or channel of the PIG 120a.

In some embodiments, the PIG 120a may comprise a sensor adapted to detect the removal of an RBS 220 (or carrier) from its internal chamber or channel. Upon detection of the removal of the RBS 220 (or carrier) from the internal chamber or channel the sensor triggers an alert system to notify a user that the radioactive source is removed from the internal chamber of the PIG 120a.

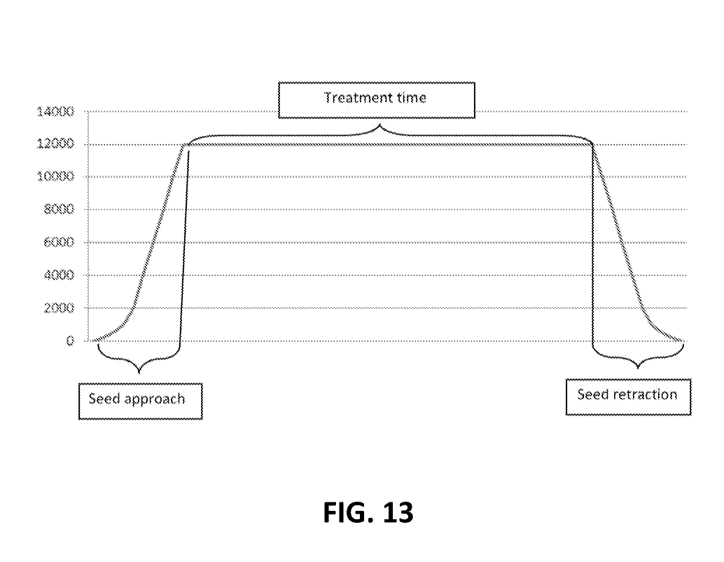

In some embodiments, the sensor is adapted to calculate a dose delivered to the treatment zone/target. In some embodiments, the senor is adapted to calculate the treatment time (e.g., the amount of time the target is exposed to the RBS 220) (see FIG. 13).

In some embodiments, the sensor may be operatively connected to both a power source and an alert system. However, the present invention is not limited to his configuration. For example, the sensor may incorporate the alert system. The sensor may not require a power source. For example, in some embodiments, the sensor is phosphorus (e.g., a non-electrical system) and upon detection of a RBS the phosphorus undergoes a reaction causing a visible change in the appearance of the phosphorus (e.g., the appearance of the phosphorus is the “alert system”). The sensor is not limited to the aforementioned non-electrical system (phosphorous). The alert system may provide a visual and/or an audio alert.

The sensor may be an electrical system. For example, the sensor may be a simple electronic circuit (e.g., with a battery). Such sensors may include but are not limited to an optical sensor (e.g., OMRON ELECTRONICS LLC Part number EE-SPX304-W2A). The optical sensor may detect when the spiral cut tube is advanced as it passes the sensor, for example the optical sensor may activate a light emitting diode (LED) when the RBS/spiral cut tube is advanced and allow the LED to remain on while the RBS/spiral cut tube is advanced (e.g., while it is in the treatment zone). When the RBS/spiral cut tube is pulled back (and the RBS is in the PIG) the LED turns off.

The sensor may be a transistor, e.g., a solid-state transistor. In some embodiments, the transistor is a metal-oxide-semiconductor field-effect transistor (MOSFET). Such transistors are well known to one of ordinary skill in the art. For example, Sicel Technologies (Morrisville, N.C.) has a p-channel MOSFET detector with a 4000 Angstrom oxide layer. The MOSFET (as well as a data acquisition chip, a microprocessor, and a copper coil) is encapsulated in a glass tube 3.25 mm in diameter and 25 mm in length. The circuit is powered by a current induced in the coil by an external handheld antenna connected to an rf reader. The dosimeter is passive during irradiation and powered only during measurement of the threshold voltage of the MOSFET. The microprocessor controls both data acquisition and reader/dosimeter communication. A computer controls the rf reader and converts the digital signal to a decimal voltage. The MOSFET is a wireless device adapted to precisely measure the dose of radiation to a specific site. In some embodiments, the sensor is an electromagnetic transponder or the like.

With the sensors, the device (e.g., cannula, PIG 120a, etc.) may be constructed from a variety of materials. For example, the PIG may be generally clear, translucent, or transparent, or the PIG 120a may be pigmented, colored, or opaque because the sensor provides indications that the RBS is in a particular location (e.g., treatment zone, in the inner chamber/channel of the PIG 120a) regardless of whether the RBS 220 can be seen in the PIG 120a or cannula. In some embodiments, the PIG 120a is constructed from a plastic, a glass (e.g., durable glass, Gorilla® Glass), or a combination thereof. In some embodiments, the PIG is constructed from a material comprising a polyetherimide, poly (methyl methacrylate), acrylic polysulfone, polycarbonate, polypropylene, stainless steel, aluminum, polyether ether ketone, or a combination thereof.

The sensors (e.g., sensor at the tip of the cannula) may be wired with a wire that runs along the cannula, similar to how the light fibers run along the handle/PIG 120. This may allow the transmission coil to be located in the PIG/handle 120, thus reducing the size of the sensor on the tip. In some embodiments, a passive sensor may be hard-wired to a monitor/control unit so as to minimize the size of the sensor and eliminate the need for signal transmission.

The sensors may be operatively connected to a power switch for turning the sensors on and off. The alert system may include a monitor designed to register the radiation dosage (dosimeters). When the RBS 220 is loaded in the device, the RBS 220 initially resides in the PIG. PIG 120a alert system/monitor can be observed to ensure that the sensor in the PIG 120a is detecting the radiation in the PIG. Upon deployment of the RBS, the alert system/monitor should show a drop in the detected radiation within the PIG 120a and an increase in the detected radiation at the tip. Dosage at the treatment zone can be monitored and measured (e.g., real-time). Upon retraction of the RBS, the alert system/monitor should show a drop in the detected radiation at the tip and an increase in the detected radiation in the PIG 120a.

Other means of verifying RBS location may be employed, such as additional devices with sensors adapted to determine the position of the RBS. For example, the cannula/handle/PIG may be inserted into a chamber with one or more sensors adapted to detect an RBS (e.g., transistors, optical sensors, chemicals, Geiger counters, etc.). The RBS can be deployed and retracted within the device and the sensors can calculate the location of the RBS, for example the sensors can determine whether the RBS reaches the intended location (e.g., treatment zone) when deployed.

As shown in FIG. 17, the present invention may also feature a switch sensor 707 (e.g., a “Radiation Optic Switch (ROS)) for detecting the presence of the RBS in a treatment zone (e.g., at the tip of the cannula). The switch sensor 707 may be a non-electric sensor (e.g., phosphorous). The switch sensor 707 is operatively connected to an alert system 708, for example via fiber optics (e.g., a “fiber”), which may run the length of the cannula from the tip past the handle. When the switch sensor 707 is activated (upon detection of the RBS in the treatment zone), the switch sensor 707 activates the alert system 708 via the fiber. In some embodiments, the alert system 708 is a switch sensor light system, wherein the switch sensor light system is illuminated when the switch sensor 707 is activated by the presence of the RBS in the treatment zone.

### Verifying/Calibrating RBS Placement

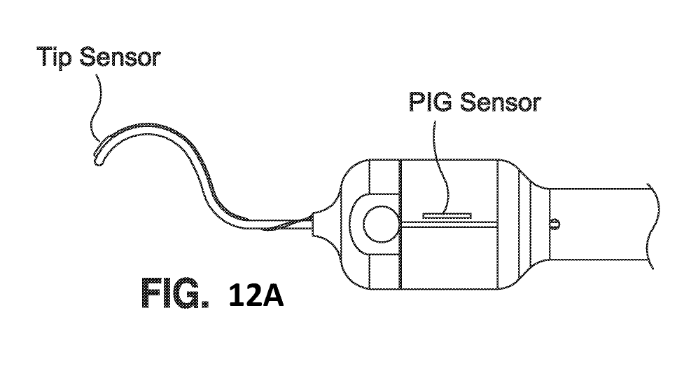

The present invention features methods and devices for verifying and calibrating RBS placement (see also Example 3 below). As shown in FIG. 11, a radiation shield may comprise a base and a lid pivotally or removably attached to the base. The lid forms an inner cavity. The lid can move between at least an open position and a closed position respectively allowing and preventing access to the inner cavity. A groove is disposed in the top surface of the base near a side edge. The groove is adapted to hold the PIG/handle 120 of the device of the present invention. A slot is disposed in the lid at the bottom surface. The slot is adapted to allow the cannula (e.g., distal portion, proximal portion) to pass into the inner cavity. The slot and the groove align when the lid is in the closed position. Generally the radiation shield is constructed to block radiation, for example the lid may be constructed from a material comprising plastic and may be of sufficient thickness to block the passing of beta radiation.

The radiation shield can be used to verify or calibrate RBS placement (e.g., placement at a treatment position in the tip of the cannula). For example, a film (e.g., GafChromic® film, dosimetry film) is placed in the inner cavity of the radiation shield. The tip of the cannula is placed atop a visual marker disposed on the film. The light source of the cannula is aligned atop the visual marker on the film. The advancing means is activated (e.g., via actuator handle, etc.) to advance the RBS to the tip of the cannula (or near the tip of the cannula) for a first length of time (e.g., 5 seconds, between about 5 to 10 seconds, more than about 10 seconds, between about 2 to 5 seconds, etc.). A reaction on the film occurs due to exposure to the RBS. The film is analyzed. If the reaction on the film occurs on the visual marker of the film, the RBS placement is calibrated. If the reaction on the film does not occur on the visual marker of the film, the RBS placement is not calibrated. RBS placement can be adjusted by adjusting the advancing means/actuator, for example via adjusting the actuator stop-collar position (e.g., with an Allen wrench).

A dose at a given depth may be calibrated in accordance with novel methods of the present invention. In some embodiments, a method of calculating the dose is a Monte Carlo simulation. This provides the relationship between depth of the target and dose deposited. For example, a film is exposed with a known spacer (e.g., 2 mm). The film is also exposed to a known dose of radiation, such as from a calibrated linear accelerator. The comparative optical density of the film exposure is used to calculate the actual dose.

The depth verses dose relationship from the Monte Carlo calculation is normalized (e.g., re-set) to the dose at depth found empirically from the film exposures. Dose at any depth is then calculated from the normalized (e.g., re-set) Monte Carlo.

For example, as the calibration standard NV 843 had an activity of 365.6 MBq, the activity of the prototype therapeutic source (SR 800) was determined to be 365.6×9.866/4.970 MBq=726 MBq=19.6 mCi Sr-90/Y-90. The radioactive Sr-90/Y-90 source is that of a prototype therapeutic source (called SR 800) drawing VZ-2911-005 in a straight cannula. The equipments used included solid water block, GafChromic film MD-55, Scanner Nikon Super Coolscan 8000ED1.11 LS800ED SN217410 with holder FH-8695, software Nikon Scan 3.1 FastV3.01. Measurements made: Care was taken that the same time elapsed between irradiation and scanning for all measurements. First measurements suffered from a small inaccuracy in the manufacture of the solid block (an air gap of approximately 200 um). This was repaired by modifying the irradiation geometry. A comparison of the optical density between films with known dose and the film irradiated with the prototype therapeutic source SR 800 in a straight cannula in 2 mm distance (in a solid water block) from the cannula midpoint for 7:03 minutes gave a dose of 62.6 Gy, this is a dose of 8.9 Gy/min in 2 mm distance (in a solid water block) from the cannula midpoint.

### Two-Piece System And Assembly

The present invention may be divided into a two-piece device, however the present invention is not limited to this configuration. The devices of the present invention may be constructed as a single piece.

In some embodiments, the device comprises a cannula subassembly 124 and a handle subassembly 125. The cannula subassembly 124 may comprise a generally hollow fixed shape cannula with a distal portion 110a for placement around a portion of a globe of an eye, a proximal portion 110b and an inflection point, which is where the distal portion 110a and the proximal portion 110b connect with each other. The proximal portion 110b may be attached to a cap/hub/connector component. The distal portion 110a may have a radius of curvature between about 9 to 15 mm and an arc length between about 25 to 35 mm. The proximal portion 110 b may have a radius of curvature between about an inner cross-sectional radius of the cannula and about 1 meter.

The handle subassembly 125 may comprise a handle 120 having a first end and a second end, the first end being adapted to removably engage the cap/hub/connector component of the cannula subassembly 124. A radiation shielding PIG 120a for shielding radiation may be disposed in the handle 120. A channel 219 may be disposed in the handle 120 and the PIG 120a, wherein the channel 219 is positioned such that the channel 219 aligns with the hollow fixed shape cannula when the first end of the handle 120 engages the cap/hub/connector component of the cannula subassembly 124. The handle subassembly 125 may further comprise an advancing means for advancing a RBS.

The cannula subassembly 124 and the handle subassembly 125 may connect together (e.g., see FIG. 10). In some embodiments, the cannula subassembly 124 attaches to the handle subassembly 125 with attachment thumb screws 320. The handle subassembly 125 may also includes the control cable 150, and the cannula subassembly 124 may also include the light fiber 180 and light connector component 195.

Without wishing to limit the present invention to any theory or mechanism, it is believed that the use of the engineering polymer (Ultem® Polyetherimide) is advantageous because it is radiation resistant and structurally durable, and is also translucent, which can provide visual access through the PIG/handle 120. For example, the polymer provides the required shielding to protect the physician prior, during and after the procedure; while the diameter allows the physician to visualize the seed's position in the handle/PIG 120 prior to deployment and after retrieval.

FIG. 3A-3C shows a channel 219 disposed in the PIG/handle adapted to hold the spiral cut tube and RBS. The spiral cut tube 210, as shown in FIG. 3B, can be seen in the channel 219, for example when the spiral cut tube/RBS 210, 220 is advanced to the treatment position. In some embodiments, either before or after treatment, the spiral cut tube/RBS 210, 220 may also be visualized in the channel 219. As shown in FIG. 3C, the end of the spiral cut tube/RBS 210, 220 is visible in the PIG/handle 120.

The flexible spiral cut tube 210 may be advantageous in the cannulas of the present invention (e.g., S-shaped cannulas). The devices of the present invention allow the cannula to contour to the curvature of the eye and the PIG/handle 120 to be directed out of the line of sight for visualization.

As the actuating handle 150 is advanced, the advancing means pushes the spiral cut tube/RBS forward from the PIG/handle 120 and down the cannula. As it encounters the first radius of the cannula, the spiral cut tube 210 is allowed to flex. The spiral cut tube 210 can recover to a straight tube in the straight segments and continues to flex as it encounters the second radius as needed.

In an embodiment the system has a light system (e.g., fiber 180) with a tip 260 to illuminate the posterior portion of the eye from the subtenon space. The light system can be activated to illuminate the macular and posterior pole landmarks such as but not limited to the macula lutea, fovea centralis, optic disc. The illumination may be designed to center about the middle of a deployed radioactive seed 220. The light fiber 180 (e.g., tip 260) may be cut at a 40-75 degree angle to direct the light into the vitreous like a lens to indicate the middle of the seed, however the light fiber (e.g., tip 260) is not limited to this configuration.

The light fiber (or group of fibers 180) is made of PMMA and is resistant to radiation exposure and can be Ethylene Oxide (ETO or EO), Gamma or Electron beam sterilized. A grouping of three fibers may be appropriate for light transmission, however the present invention is not limited to this configuration. The fibers run along the outside of the cannula (e.g., distal/proximal portions), which may then be covered with a PET (polytheylene terephthalate) outer heat shrink tube. The fibers may also be tacked in place along the length of the cannula with a UV adhesive to aid in the fixation.

A control cable 150 for lightweight flexible control would not interfere with the positioning of the device. The control cable 150 may also not contribute to fatigue of the physician's hand due. In some embodiments, a control cable 150 can be designed like that of a camera. In some embodiments, the control cable 150 features a central wire rope made of 300 series stainless steel with a 7×19 strand core and a nylon-coating with an outer tube polyvinyl chloride (PVC) lined with FEP Teflon on the ID having a durometer of about 67 A. The nylon and Teflon interaction may provide a low surface friction, allowing the tubes to slide easily. A stainless steel tube added to the length of the throw may improve the column strength of the central wire rope that attaches to the deployment plunger ring; in the only area it was encapsulated.

Since the cannula subassembly 124 may be removable post surgery from the handle subassembly 125, radiation has the potential to escape. Therefore in some embodiments, a secondary radiation shield 128 is used as a PIG cover to attach to the end of the handle subassembly 125 (see FIG. 9). The secondary radiation shield 128 may be constructed from a material comprising Ultem® (based on the same shielding data used for the PIG/handle 120 to safely store the seed within the PIG/handle 120).

### Spacing Between Radiation Source And Sclera

The present invention also features a cannula tip comprising a radiation source that is spaced at a fixed distance away from the sclera. In some embodiments, the cannula tip is located at or near the distal end of the distal portion 110a of the cannula. The distance between the radiation source closest to the sclera and the sclera itself is between about 0.1 mm to about 1 cm, e.g., about 0.1 to 0.5 mm, 0.5 mm to 1.0 mm, 1.0 mm to 3 mm, 3 mm to 1.0 cm. The surprising result of providing for this distance between the radiation source and the sclera is that the sclera receives less radiation dose, but yet the target inside the eye receives a dose that is substantially unchanged as compared to when the radiation source is on the sclera (i.e., when the radiation source is not spaced at a fixed distance away from the sclera). In some embodiments, the radiation source used in accordance with the present invention includes Sr-90/Y-90 and P-32 sources.

### Spacing Between Radiation Source And Sclera Is Filled By A Material

The spacing between the radiation source and the sclera may comprise of anything that is appropriate (e.g., vacuum, gas, liquid and/or solid). In some embodiments, a solid material provides for a fixed spacing between the radiation source and the sclera. In some embodiments, the solid materials that may be used in accordance with the present invention include stainless steel, titanium, aluminum, composite materials such as PET and PEEK, and the like.

When selecting an beta emitter to use as a brachytherapy source, it is surprising that the water equivalent thickness of metals such as stainless steel, titanium, aluminum, composite materials such as PET, PEEK, and other substances for the beta radiation emitted by P-32, which has a mean beta particle energy of 0.695 MeV is smaller than for the beta radiation emitted by sources having higher mean beta particle energies up to approximately 4 MeV, such as Y-90, Sr-90/Y-90, Ru-106. Accordingly, one of the surprising advantages of using steel is that, for the subset use of when it is used with a P-32 source, steel does not stop as much of the P-32 as compared to Sr-90.

### Materials Used In Accordance With the Present Invention

Ultem® 1000/PEI (Polyetherimide), http://machinedesign.com/article/polyetherimide-1115

The source activity is about 10 mCi. When positioning the brachytherapy device, the surgeon may grip it with fingers placed on the exterior of the region of the seed shielding. It is during this time that the surgeon receives dose. Once the source is deployed for treatment, the dose received by the surgeon is negligible. The surgeon may hold the device for 10 minutes per procedure before deploying the source.

### Calculations

Source Description: Sr-90 is a pure beta emitter with a maximum beta particle energy of 0.546 MeV. It decays into Y-90 with a half-life of 28.8 years. Y-90 is a pure beta emitter with a half-live of 64 h, maximum beta energy of 2.28 MeV and mean beta energy of 0.935 MeV. Since the beta particles emitted by Y-90 are more energetic than those emitted by Sr-90, the delivered dose is nearly all attributable to Y-90. Furthermore, the activity of the Y-90 is in equilibrium with Sr-90.

The dose rate of the Sr-90 seed is 1.1 Gy/min/(mCi) at a depth of 2 mm and, therefore 1.767 times this value at a depth of 1.5 mm (AAPM Task Group 149 Table IX and Table XII). The relationship between the target point dose, D, treatment time, T, and the source activity, A, is

$$\frac{D}{T} = 1.9437 A ( \frac{Gy / min}{mCi} )$$

For example, to deliver 24 Gy at a depth of 1.5 mm in water, ignoring attenuation of the radiation by the device, with a 10 mCi source requires 1.9 minutes.

### Surgeon Dose

The total dose received by the surgeon during a procedure depends on the amount of time that the device is held without deploying the source and on the source activity, since the surgeon does not receive dose once the source is deployed. The higher the activity, the more dose received by the surgeon. The time that it takes to place the device is independent of the source activity, so there may be a tradeoff between short treatment delivery time and surgeon dose. Shorter delivery times require higher source activities, which may result in a higher dose to the surgeon. The shielding used will be enough to stop all of the beta particles (electrons) emitted during the Sr-90/Y-90 decays. However, when electrons are stopped, they produce bremsstrahlung radiation, which is high energy x-ray photons. These photons will travel a substantial distance in the stopping material. In fact, it is not possible to shield the user from all of these x-rays. Thus, it is the x-rays resulting from the stopping of the beta rays (electrons) that result in radiation dose to the surgeon.

The dose rate received is:

$$\dot{D} = ( \frac{{μ}_{en}}{ρ} ) {Ψ}_{γ}$$

where {·μₑₙ I p) is the mass energy absorption coefficient and ψ_{y} is the photon energy flux. The value of {·μₑₙ I p) for water is used when calculating the dose to the surgeon's hand, making the assumption that the dose to the hand tissue is close to that of water. This is routine in radiation therapy dosimetry. Using the actual value for tissue would differ by less than 2%.

The photon energy flux, ψy=Photon Energy/[sec cm²]=Yψₑ where Yψₑ is the electron energy flux and Y is the bremsstrahlung yield for the stopping material.

Y=[Bremsstrahlung Photon Energy I Electron Energy].

$${Ψ}_{e} = \frac{{AE}_{D}}{4 π {r}^{2}} = Electron Energy / [ sec {cm}^{2} ] ,$$

where r is the distance for the source. A point source approximation is used for the estimate presented here. The electron energy emitted per second=AE_{D} where A is the source activity and E_{D} is the mean electron energy per decay of the radiation source (0.935 MeV for Sr-90/Y-90 seed sued in the device considered here).

The attenuation of the photon energy flux, ψ_{γ} is

$${Ψ}_{γ} ( depth = d ) = \frac{{Ψ}_{γ} ( depth = d ) {e}^{- μ d} {r}_{( d = 0 )}^{2}}{{r}_{( d = d )}^{2} {Ψ}_{γ} ( depth = d )}$$

where μ is the linear attenuation coefficient of the absorbing material.

Published values of bremsstrahlung yield, Y, and the range of electrons can be found in International Commission on Radiation Units and Measurements (ICRU) Report 37, “Stopping Powers for Electrons and Positrons” by David K. Brice (1984) and on the National Institute of Standards and Technology (NIST) website ©2010. The values for polyetherimide (Ultem®) are used herein.

Published values of {·μₑₙ I p), the mass energy absorption coefficient, and the linear attenuation coefficient can be found in Seltzer, S. M., Calculation of Photon Mass Energy-Transfer and Mass Energy-Absorption Coefficients, Rad. Res. 136, 147-170 (1993), and on the National Institute of Standards and Technology (NIST) website ©2010.

Polyetherimide (Ultem®) has a density of 1.27 g/cc. Its radiological properties are determined by its repeat unit, C₃₇H₂₄O₆N₂, having a molecular weight of 592.6 g/mol.

The ESTAR NIST database was used to find the properties. The range of the maximum energy electrons (2.28 MeV) is 0.96 cm and the bremsstrahlung yield, Y=0.00749. The range of the mean energy electrons is 0.34 cm and Y=0.00307. Thus, 1.0 cm of polyetherimide is enough to stop the electrons, but some x-rays will be produced.

If an additional 0.1 cm of polyetherimide were used, making the thickness 1.1 cm, then using the above equations, the dose received by the surgeon per 10 minute procedure would be <0.01 mSv. This calculation is a conservative estimate as it assumes that the mean bremsstrahlung x-ray energy is 0.9 MeV.

A polyetherimide cylinder having an outside diameter of 2.3 cm and a length of 2.2 cm containing a hole drilled through the symmetry axis of diameter 50.1 cm will provide shielding such that when using a 10 mCi Sr-90/Y-90 source, a hand dose of ≦0.01 mSv will be received if the device is held for 10 minutes at the point of shielding.

The NRC limits the annual dose received by radiation workers to 50 mSv total body and 500 mSv to an extremity. Therefore, a surgeon could perform approximately 50,000 procedures per year with such a device.

In some embodiments, a fiber such as a poly(methyl methacrylate) acrylic fiber is used, for example a 0.010″ diameter and NA.5 from Fiberoptics Technology, Incorporated (Pomfret, Conn.) is used. Information about the fibers can be found on the company's website ©2001. In some embodiments, a fiber for low heat or high heat (e.g., melt resistant to 70 degrees Celsius, for example) may be used.

The source of the above graphs (Graph 3-05 and Graph 3-07) is The Effect of Sterilization Methods on Plastics and Elastomers by Liesl K. Massey, 2^{nd} Edition, copyright 2005 by William Andrew, Incorporated (Norwich, N.Y., USA).

PET (Polyethylene terephthalate) for heat shrink tubing may be, for example, obtained from Advanced Polymers, Incorporated (Salem, New Hampshire). Information about the PET from Advanced Polymers, Incorporated can be found on the company's website ©2010. In some embodiments, the heat shrink tubing can be sterilized using ethylene oxide, gamma radiation, E-Beam, or autoclaving.

Alternatively material to PET is PEEK (Poly ether ether ketone), for example PEEKshrink® from Zeus® (Orangeburg, S.C.). PEEK is not greatly resistant to UV radiation but has good resistance to beta and X-rays, as well as exceptional resistance to gamma rays (more than 1000 Mrad without significant loss in mechanical properties). These properties allow for ease of sterilization, and coupled with good biocompatibility (USP Class VI).

Stainless steel 300 series and Nitinol neither are impacted by these levels of radiation.

Materials that may be used in accordance with the present invention (e.g., wherein the material is not going to interact with the radioactive seed) may include but are not limited to: a fluorinated ethylene polypropylene (FEP)-lined PVC tube or stainless steel for the cable jacket, for example from McMaster Carr® (Elmhurst, Ill.), for example part number 5046K11; a nylon coated stainless steel for the control cable, for example from McMaster Carr® (Elmhurst, Ill.), for example part number 34235T29; a silicone O-ring for the o-ring, for example from McMaster Carr® (Elmhurst, Ill.), for example part number 9396K16; and polycarbonate light source adaptors. A light source may include but is not limited to a Scintillant® surgical light from Engineering Medical Solutions (Phillipsburg, N.J.), for example part number 2658-01-001 (information about part number 2658-01-001 can be found on the company's website ©2009).

### Alternative Materials

One alternative embodiment would be to replace the Ultem® with another translucent polymer that can be sterilized and would create a radioactive shield (PIG). Another polymer that was explored was Lucite, but it is believed that other polymers are potential candidates such as but not limited to Polysulfone, Polycarbonate, Polypropylene.

### Using Lucite As A Radiation Shield

Iron (Stainless Steel): The range of the maximum energy electrons (2.28 MeV) is 0.2 cm and the bremsstrahlung yield, Y=0.035. The range of the mean energy electrons is 0.07 cm and Y=0.0156. Thus, 0.2 cm of iron is enough to stop the electrons, but a substantial amount of x-rays will be produced. If an additional 0.9 cm of iron were used, then using the above equations, the dose received by the surgeon per 10 minute procedure would be approximately 1 mSv, assuming that the mean bremsstrahlung x-ray energy is 0.9 MeV. This calculation is a conservative estimate.

Lucite: The range of the maximum energy electrons (2.28 MeV) is 0.97 cm and the bremsstrahlung yield, Y=0.00723. The range of the mean energy electrons is 0.33 cm and Y=0.003. Thus, 1.0 cm of Lucite is enough to stop the electrons and only a minimal amount of x-rays will be produced. If an additional 0.1 cm of Lucite were used, then using the above equations, the dose received by the surgeon per 10 minute procedure would be 0.2 mSv, assuming that the mean bremsstrahlung x-ray energy is 0.9 MeV. This calculation is a conservative estimate. Since Lucite is lighter and provides better shielding for a thickness of 1.1 cm, it is preferred over lead.

Summary: A therapeutic dose of radiation can be delivered in 1.54 minutes using a 10 mCi an FDA approved Sr-90/Y-90 source. A Lucite cylinder having an outside diameter of 2.3 cm and a length of 2.2 cm containing a hole drilled through the symmetry axis of diameter ≦0.1 cm will provide shielding such that using a 10 mCi Sr-90/Y-90 source, a hand dose of ≦0.2 mSv will be received if the device is held for 10 minutes at the point of shielding. The NRC limits the annual dose received by radiation workers to 50 mSv total body and 500 mSv to an extremity. Therefore, a surgeon could perform approximately 2,500 procedures per year with such a device.

Another alternative embodiment similar to the aforementioned stainless steel could be used as a PIG. Other materials that may be used include but are not limited to aluminum, titanium, elgiloy and lead. These materials would not have the translucent properties but they that compromise would enable the addition of the other parts of this invention to be realized.

### Alternative Cannula Shape And Material

Cannula shape in the embodiment may be a round 16 gauge hypodermic need tube, however the size range can vary from 10-22 gauge and the shape does not have to be a diameter at all. It can have a cross section that include but are not limited to an oval, square, diamond, rounded rectangle, and a hexagon, etc.

### Alternative Light

As PMMA may be used in accordance with the present invention as a fiber material, glass fibers that are custom formed tipped may work as well, with the added advantage of being autoclavable.

An alternative means of angling the light with a straight fiber into the vitreous may include a lens or system of lenses (similar to that found on the end of an arthroscope from Karl Storz (Tuttlingen, Germany), and information about such arthroscopes can be found on the Karl Storz website © Copyright KARL STORZ GmbH & Co. KG, Tuttlingen as of October 2010. Alternative means of angling the light may also include but is not limited to a reflector or mirror assembly, for example a sapphire lens is used on a Panoview arthroscope from Richard Wolf (Vernon Hills, Ill.). The cannula or the light fiber(s) themselves may be attached to the means of angling the light, for example.

### Alternative Polymers To PET Heat Shrink Tubing

It is believed that other polymers (heat shrink tubing) can cover the cannula assembly including but not limited to PEEK (Poly ether ether ketone) heat shrink tubing, for example PEEKshrink® from Zeus® (Orangeburg, S.C.), and radiation resistant heat shrink tubing (e.g., a copolymer of ethylene and tetrafluoroethylene), for example NEOFLON™ ETFE from Daikin (Decatur, Ala. and Osaka, Japan), for example part number EP-521 (information regarding part number EP-521 is disclosed by Daikin on their website (©2005)).

### Alternative Embodiments Within the Control Cable

Alternative means for the central wire rope can be done with any elastic or super elastic member with the column strength to overcome the friction of the outer control cable tube, the flexible spiral cut tube within the S-shaped cannula, such as but not limited to a Nitinol, elgiloy, or combination rope. Of various tempers and/or material states as well know to those skilled in the art.

Alternative means of coating may be applied to reduce friction, such as but not limited to PTFE, FEP and Acrylic. Surface treatments may also be applied such as but not limited to Diamond-like carbon and Titanium nitride. Such materials may be found from Morgan Technical Ceramics Diamonex (Berkshire, England) and NCT Coating (Manitoba, Canada).

Alternative means for the outer tube may be a soft tube such as but not limited to Pebax, Nylon, Polyester lined with Teflon on the ID. Alternative means for the outer tube would be a soft tube such as but not limited to Pebax, Nylon, Polyester coextruded with Teflon on the ID.

### Alternative Embodiments Within The Illumination Connector/Light Connecting Component (Materials And Design)

The light source adaptors are black polycarbonate to reduce cost and residue light that might escape into the surgical field other materials is used to obtain the same goal such as but not limited to, Ultem®, Delrin, light blocking pigmented polymers that are well know to those skilled in the art. Additionally metals or ceramic can be used, such as but not limited to: aluminum, stainless steel, and other that are well know to those skilled in the art.

ID can be textured to increase the frictional fit over the length of the illumination connector, and lock the light source in place.

The light source adaptors o-ring is silicone but is made of any material that would provide the appropriate holding friction such as but not limited to: Buna-N, Latex, Ethylene-Propylene, Polyurethane, Neoprene®, Fluorocarbon, and Fluorosilicone.

An alternate design to the o-ring can be to add a layer of polymer similar to the polymers used for the o-rings to line the inside diameter of the illumination connector to achieve an uninterrupted frictional fit over the length of the illumination connector. This would meet the requirements of the to hold and lock the device in place at any given distance along the light source focal point within the illumination connector with respect to the light fibers.

### EXAMPLE 1

### Surgical Procedure

The following example describes a surgical procedure using a device of the present invention.

1. Create buttonhole incision in conjunctiva and Tenon's Capsule.

2. Gently separate Tenon's Capsule from sclera by injecting Balance Salt Solution (BSS) or lidocaine without epinephrine.

3. Insert the distal tip of the cannula into the subtenon space using the previously made incisions.

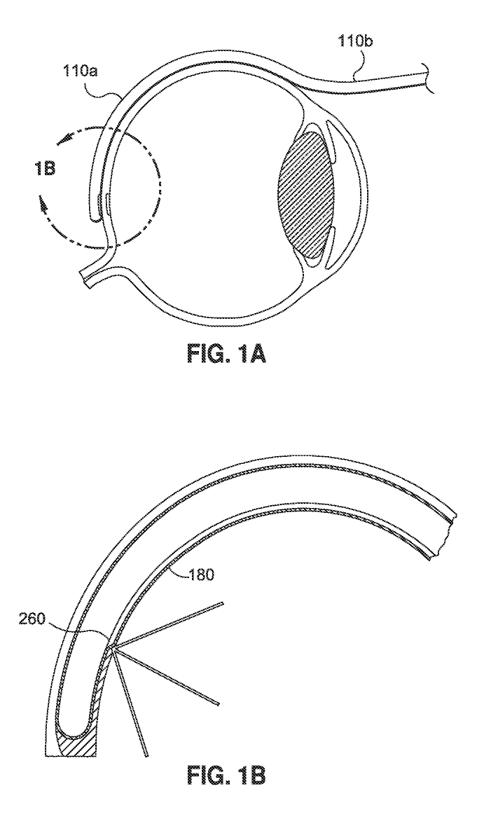

4. Continue to insert the cannula until the distal tip is located at the posterior pole of the eye (See FIG. 1A).

5. Once this gross position is achieved, activate the light tip of the cannula by activating the power of the light source.

6. View the lighted tip through an indirect ophthalmoscope and adjust the tip position for treatment of the defect (see FIG. 1B).

7. Brachytherapy administration: Deploy the radioactive seed. Full deployment of the seed may be verified visually in the handle 120 of the device (e.g., viewing a visual marker on the plunger, viewing a visual marker on the RBS/spiral cut tube/solid shaft, etc.).

8. Leave in place for the predetermined treatment time.

9. Retract the seed (e.g., by pulling the actuator handle) back to the original position. Full retraction of the seed may be verified visually in the handle 120 of the device (e.g., viewing a visual marker on the plunger, viewing a visual marker on the RBS/spiral cut tube/solid shaft, etc.).

10. Remove cannula from subtenon space.

### EXAMPLE 2

### Calibration of RBS Placement

The following example describes an example of verifying/calibrating RBS placement using GafChromic® film (e.g., procedure for checking deployment of the seed, adjusting the travel of the seed in the device to center it under the light source fiberoptic termination). Testing was performed within an acrylic test box (e.g., radiation shield with inner cavity).

1. A pen mark was placed on the GafChromic® film. The film and device (loaded with a radioisotope seed) were placed into the box.

2. The cannula tip light was centered on the pen mark. The seed was then deployed. In approximately 5 seconds, a small dark exposure was noted.

3. Examination showed that the exposure was not centered on the pen mark. Thus, the device actuator stop-collar position was adjusted with an Allen wrench.

4. The cannula tip light was again centered on another mark on the film. The seed was then re-deployed. In approximately 5 seconds, a small dark exposure was noted. This was found to be centered on the pen mark. This was a successful test confirming deployment of the seed and also confirming adjusting the position of the seed so as to center on the light source.

### EXAMPLE 3

### Testing of Sterilization of Film

The following example describes a test of the competency of ethylene oxide (EO) sterilized GafChromic® film.

1. Previously GafChromic® film was packaged and EO sterilized in a sterile pouch.

2. The GafChromic® film was inspected inside its sterile pouch. The film appeared without evidence of damage. It was noted that the EO indicator strip had turned positive.

3. Testing was performed within an acrylic test box (radiation shield with inner cavity). The device cannula was placed over the outside of the sterilization pouch. The seed was advanced. In approximately 5 seconds, a small dark exposure was noted.

4. Following the procedure the sterile package was opened and the film directly inspected. No discoloration nor other damage was noted. It was concluded that the GafChromic® film retained its competency for this purpose following EO sterilization.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims.

1. A brachytherapy device comprising a handle having a radiation shielding PIG for shielding a RBS, at least a portion of the radiation shielding PIG is generally visually clear, transparent, or translucent.

2. The brachytherapy device of claim 1, wherein the portion of the radiation shielding PIG that generally visually clear, transparent, or translucent is a window.

3. The brachytherapy device of claim 1, wherein the handle is constructed from a material comprising plastic, glass, a polyetherimide, poly (methyl methacrylate), acrylic polysulfone, polycarbonate, polypropylene, or a combination thereof.

4. The brachytherapy device of claim 1 further comprising a distal portion for placement around a portion of a globe of an eye, the distal portion has a radius of curvature between about 9 to 15 mm and an arc length between about 25 to 35 mm; a proximal portion having a radius of curvature between about an inner cross-sectional radius of the cannula and about 1 meter; and an inflection point which is where the distal portion and the proximal portions connect with each other; wherein the handle is attached to the proximal portion.

5. The brachytherapy device of claim 1, wherein the radiation shielding PIG is constructed from a material comprising a polyetherimide, polysulfone, polycarbonate, or polypropylene.

6. The brachytherapy device of claim 1, wherein a visual landmark is disposed on the PIG, the visual landmark functions as a reference point for orientation before or during a surgical procedure.

7. The brachytherapy device of claim 1 further comprising a means of moving a RBS within the handle.

8. The brachytherapy device of claim 7 further comprising a control cable system for manipulating the means of moving the RBS.

9. The brachytherapy device of claim 8, wherein an actuator handle is disposed on a second end of the control cable system.

10. (canceled)

11. (canceled)

12. The brachytherapy device of claim 1 further comprising a secondary radiation shield attachable to the handle.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

35. (canceled)

37. (canceled)

38. A cannula comprising a sensor for detecting a presence of an RBS at a position within the cannula, the sensor is operatively connected to both a power source and an alert system, wherein upon detection of the presence of the RBS at the position within the cannula the sensor triggers the alert system to notify a user that the RBS is at the position within the cannula.

39. The cannula of claim 38, wherein the sensor detects the presence of the RBS in a treatment zone.

40. The cannula of claim 38, wherein the sensor activates a light source when the RBS is detected in the treatment zone.

41. The cannula of claim 38, wherein the sensor is an electrical system.

42. (canceled)

43. (canceled)

44. (canceled)

45. (canceled)

46. (canceled)

47. (canceled)

48. (canceled)

49. (canceled)

50. (canceled)

51. (canceled)

52. A PIG having an internal chamber, the PIG comprising a sensor adapted for (i) detecting presence of a radioactive source or a carrier within the internal chamber, (ii) detecting removal of a radioactive source or a carrier within the internal chamber, or (iii) both detecting presence of a radioactive source or a carrier within the internal chamber and detecting removal of the radioactive source or the carrier within the internal chamber, the sensor is operatively connected to both a power source and an alert system, wherein upon detection of presence of the radioactive source or the carrier within the internal chamber the sensor triggers the alert system to notify a user that the radioactive source is within the internal chamber of the or wherein upon detection of removal of the radioactive source or the carrier within the internal chamber the sensor triggers the alert system to notify a user that the radioactive source is removed from the internal chamber of the PIG.

53. The PIG of claim 52 wherein the sensor can detect the presence of a mass being stored within the internal chamber, the mass includes a radioactive source.

54. The PIG of claim 32 wherein the alert system provides a visual alert.

55. The PIG of claim 52 wherein the alert system provides an audio alert.

56. The PIG of claim 52 wherein the sensor is an optical sensor.

57. The PIG of claim 52 wherein the sensor is an electrical system.

58. (canceled)

59. (canceled)

60. (canceled)

61. (canceled)

62. (canceled)
